(19) United States
(12) Patent Application Publication
(10) Pub. No.: US 2018/0117210 A1
(43) Pub. Date: May 3, 2018

(54) SOLID DRESSING FOR TREATING WOUNDED TISSUE AND PROCESSES FOR MIXING FIBRINOGEN AND THROMBIN WHILE PRESERVING FIBRIN-FORMING ABILITY, COMPOSITIONS PRODUCED BY THESE PROCESSES, AND THE USE THEREOF

(71) Applicant: STB, LTD., BELLEVUE, WA (US)

(72) Inventors: MARTIN MACPHEE, DARNESTOWN, MD (US); DAWSON BEALL, GAITHERSBURG, MD (US); DANIEL GRAHAM, GREENWOOD VILLAGE, CO (US); ROB MARTEL, GREENWOOD VILLAGE, CO (US); ANGELA MITCHEL, GREENWOOD VILLAGE, CO (US); CHRISTINE HAEFLING, GREENWOOD VILLAGE, CO (US); PETER BROWN, GREENWOOD VILLAGE, CO (US); RICH DEGERONIMO, GREENWOOD VILLAGE, CO (US); JERRY KANELLOS, ELTHAM (AU); BELINDA WILMER, MARTINSBURG, WV (US); SHIRLEY MIEKKA, COLORADO SPRINGS, CO (US)

(21) Appl. No.: 15/605,660

(22) Filed: May 25, 2017

Related U.S. Application Data
(63) Continuation-in-part of application No. 15/208,563, filed on Jul. 12, 2016, now abandoned.
Application No. 15/208,563 is a continuation-in-part of application No. 15/208,591, filed on Jul. 12, 2016, now abandoned.
Application No. 15/208,591 is a continuation-in-part of application No. 15/088,438, filed on Apr. 1, 2016, now abandoned.
Application No. 15/088,438 is a continuation-in-part of application No. 14/884,333, filed on Oct. 15, 2015, now abandoned.
Application No. 15/088,438 is a continuation of application No. 14/583,002, filed on Dec. 24, 2014, now abandoned.
Application No. 14/583,002 is a continuation of application No. 13/364,837, filed on Feb. 2, 2012, now abandoned.
Application No. 13/364,837 is a continuation of application No. 11/882,879, filed on Aug. 6, 2007, now abandoned.
Application No. 15/208,563 is a continuation of application No. 14/746,482, filed on Jun. 22, 2015, now abandoned.
Application No. 14/746,482 is a continuation of application No. 13/364,762, filed on Feb. 2, 2012, now abandoned.
Application No. 13/364,762 is a continuation of application No. 11/882,874, filed on Aug. 6, 2007, now abandoned.
Application No. 15/208,591 is a continuation of application No. 14/599,519, filed on Jan. 18, 2015, now abandoned.
Application No. 14/599,519 is a continuation of application No. 13/363,489, filed on Feb. 1, 2012, now abandoned.
Application No. 13/363,489 is a continuation of application No. 11/882,876, filed on Aug. 6, 2007, now abandoned.
(60) Provisional application No. 60/835,423, filed on Aug. 4, 2006.
(60) Provisional application No. 60/835,423, filed on Aug. 4, 2006.
(60) Provisional application No. 60/835,423, filed on Aug. 4, 2006.
(60) Provisional application No. 62/064,291, filed on Oct. 15, 2014.

(51) Int. Cl. A61L 15/64; A61F 13/02; A61L 15/44; A61K 38/36; A61K 38/48; A61L 15/58; A61L 15/38; A61L 15/26; A61F 13/00; A61L 15/18; A61L 15/28; A61L 15/32; A61L 26/00; A61L 15/22; C08L 89/00

(52) U.S. Cl. CPC A61L 15/64; A61F 13/0246; C08L 89/00; A61L 15/44; A61K 38/363; A61K 38/4833; A61L 2400/04; A61L 15/58; A61L 15/38; C12Y 304/21005; A61L 15/26; A61L 2300/606; A61F 13/00029; A61L 15/18; A61L 2300/608; A61L 15/28; A61L 15/325; A61L 2300/10; A61L 2300/604; A61L 2300/418; A61L 2300/254; A61L 2300/252; A61L 26/009; A61L 26/0066; A61F 13/0226; A61F 13/00012; A61F 13/00017; A61F 13/00021; A61L 26/0052; A61L 26/0042; A61L 15/32; A61L 15/225; A61F 2013/00931; A61F 2013/0091; A61F 2013/0054; A61F 2013/00536; A61F 2013/00472; A61F 2013/00174; A61F 2013/00106; A61F 13/00991; A61F 13/00068; A61F 13/00063; A61F 13/00034; A61F 13/02

(57) ABSTRACT

Disclosed are solid dressings for treating wounded tissue in mammalian patients, such as a human, comprising a haemostatic layer consisting essentially of a fibrinogen component and a fibrinogen activator, wherein the haemostatic layer(s) is cast or formed from a single aqueous solution containing the fibrinogen component and the fibrinogen activator. Also disclosed are methods for treating wounded tissue using these dressings and frozen compositions useful for preparing the haemostatic layer(s) of these dressings.

### CROSS-REFERENCE TO RELATED APPLICATIONS

The present Continuation in Part is a continuation of and claims priority to U.S. patent application Ser. No. 15/088,438, U.S. patent application Ser. No. 15/208,563, U.S. patent application Ser. No. 15/208,591, and U.S. patent application Ser. No. 14/884,333, and priority to each and all of the applications to which they in turn claim priority (as set forth below), each of which is incorporated herein by reference as if fully set forth herein.

U.S. patent application Ser. No. 15/088,438, from which this Continuation in Part claims priority, is a continuation of U.S. patent application Ser. No. 14/583,002, entitled, “Solid Dressing for Treating Wounded Tissue,” filed Dec. 24, 2012, which is a continuation of U.S. patent application Ser. No. 13/364,837, entitled, “Solid Dressing for Treating Wounded Tissue,” filed Feb. 2, 2012, which is a continuation of U.S. patent application Ser. No. 11/882,879, entitled, “Solid Dressing for Treating Wounded Tissue,” filed Aug. 6, 2007, which claims priority to U.S. Provisional Patent Application Ser. No. 60/835,423 entitled “Processes for mixing fibrinogen and thrombin under conditions that minimize fibrin formation while preserving fibrin-forming ability, compositions produced by these processes, and the use thereof” filed Aug. 4, 2006, each of which is incorporated herein by reference.

U.S. patent application Ser. No. 15/208,563, from which this Continuation in Part also claims priority, is a continuation of U.S. patent application Ser. No. 14/746,482 entitled “Solid Dressing for Treating Wounded Tissue” filed Jun. 22, 2015, which is a continuation of U.S. patent application Ser. No. 13/364,762 entitled “Solid Dressing for Treating Wounded Tissue” filed Feb. 2, 2012, which is a continuation of U.S. patent application Ser. No. 11/882,874 entitled “Solid Dressing for Treating Wounded Tissue” filed Aug. 6, 2007, which also claims priority to U.S. Provisional Patent Application Ser. No. 60/835,423 entitled “Processes for mixing fibrinogen and thrombin under conditions that minimize fibrin formation while preserving fibrin-forming ability, compositions produced by these processes, and the use thereof” filed Aug. 4, 2006, each of which is incorporated herein by reference.

U.S. patent application Ser. No. 15/208,591, from which this Continuation in Part also claims priority, is a continuation of U.S. patent application Ser. No. 14/599,519, entitled, “Solid Dressing for Treating Wounded Tissue,” filed Jan. 18, 2015, which is a continuation of U.S. patent application Ser. No. 13/363,489, entitled, “Solid Dressing for Treating Wounded Tissue,” filed Feb. 1, 2012, which is a continuation of U.S. patent application Ser. No. 11/882,876, entitled, “Solid Dressing for Treating Wounded Tissue,” filed Aug. 6, 2007, which also claims priority to U.S. Provisional Patent Application Ser. No. 60/835,423 entitled “Processes for mixing fibrinogen and thrombin under conditions that minimize fibrin formation while preserving fibrin-forming ability, compositions produced by these processes, and the use thereof” filed Aug. 4, 2006, each of which is incorporated herein by reference.

U.S. patent application Ser. No. 14/884,333, from which this Continuation in Part also claims priority, claims priority to U.S. Provisional Patent Application Ser. No. 62/064,291 entitled “Processes for Mixing Fibrinogen and Thrombin, Compositions Produced By These Processes, And The Use Thereof” filed Oct. 15, 2014, which is incorporated herein by reference.

### BACKGROUND OF THE INVENTION

The present invention relates to a solid dressing for treating wounded tissue in a mammalian patient, such as a human. The materials and methods available to stop bleeding in prehospital care (gauze dressings, direct pressure, and tourniquets) have, unfortunately, not changed significantly in the past 2000 years. See J. L. Zimmerman et al., Great Ideas in the History of Surgery (San Francisco, Calif.: Norman Publishing; 1993), 31. Even in trained hands they are not uniformly effective, and the occurrence of excessive bleeding or fatal hemorrhage from an accessible site is not uncommon. See J. M. Rocko et al., J. Trauma 22:635 (1982).

Mortality data from Vietnam indicates that 10% of combat deaths were due to uncontrolled extremity hemorrhage. See SAS/STAT Users Guide, 4th ed. (Cary, N.C.: SAS Institute Inc.; 1990). Up to one third of the deaths from ex-sanguination during the Vietnam War could have been prevented by the use of effective field hemorrhage control methods. See SAS/STAT Users Guide, 4th ed. (Cary, N.C.: SAS Institute Inc.; 1990).

Although civilian trauma mortality statistics do not provide exact numbers for prehospital deaths from extremity hemorrhage, case and anecdotal reports indicate similar occurrences. See J. M. Rocko et al. These data suggest that a substantial increase in survival can be affected by the pre-hospital use of a simple and effective method of hemorrhage control.

There are now in use a number of newer haemostatic agents that have been developed to overcome the deficiencies of traditional gauze bandages. These haemostatic agents include the following:
Microporous polysaccharide particles (TraumaDEX®, Medafor Inc., Minneapolis, Minn.);
Zeolite (QuikClot®, Z-Medica Corp, Wallington, Conn.);
Acetylated poly-N-acetyl glucosamine (Rapid Deployment Hemostat™ (RDH), Marine Polymer Technologies, Danvers, Mass.);
Chitosan (HemCon® bandage, HemCon Medical Technologies Inc., Portland Oreg.);
Liquid Fibrin Sealants (Tisseel VH, Baxter, Deerfield, Ill.)
Human fibrinogen and thrombin on equine collagen (TachoComb-S, Hafslund Nycomed Pharma, Linz, Austria);
Microdispersed oxidized cellulose (M●doc™, Alltracel Group, Dublin, Ireland);
Propyl gallate (Hemostatin™, Analytical Control Systems Inc., Fishers, Ind.);
Epsilon aminocaproic acid and thrombin (Hemarrest™ patch, Clarion Pharmaceuticals, Inc.);
Purified bovine corium collagen (Avitene® sheets (non-woven web or Avitene Microfibrillar Collagen Hemostat (MCH), Davol, Inc., Cranston, R.I.);
Controlled oxidation of regenerated cellulose (Surgicel®, Ethicon Inc., Somerville, N.J.);
Aluminum sulfate with an ethyl cellulose coating (Sorbastace Microcaps, Hemostace, LLC, New Orleans, La.);
Microporous hydrogel-forming polyacrylamide (BioHemostat, Hemodyne, Inc., Richmond Va.); and
Recombinant activated factor VII (NovoSeven®, NovoNordisk Inc., Princeton, N.J.).
   These agents have met with varying degrees of success when used in animal models of traumatic injuries and/or in the field.

One such agent is a starch-based haemostatic agent sold under the trade name TraumaDEX™. This product comprises microporous polysaccharide particles that are poured directly into or onto a wound. The particles appear to exert their haemostatic effect by absorbing water from the blood and plasma in the wound, resulting in the accumulation and concentration of clotting factors and platelets. In two studies of a lethal groin wound model, however, this agent showed no meaningful benefit over standard gauze dressings. See McManus et al., Business Briefing: Emergency Medical Review 2005, pp. 76-79 (presently available on-line at www.touchbriefings.com/pdf/1334/Wedmore.pdf).

Another particle-based agent is QuickClot™ powder, a zeolite granular haemostatic agent that is poured directly into or onto a wound. The zeolite particles also appear to exert their haemostatic effect through fluid absorption, which cause the accumulation and concentration of clotting factors and platelets. Although this agent has been used successfully in some animal studies, there remains concern about the exothermic process of fluid absorption by the particles. Some studies have shown this reaction to produce temperatures in excess of 143° C. in vitro and in excess of 50° C. in vivo, which is severe enough to cause third-degree burns. See McManus et al., Business Briefing: Emergency Medical Review 2005, at 77. The exothermic reaction of QuikClot™ has also been observed to result in gross and histological tissue changes of unknown clinical significance. Acheson et al., J. Trauma 59:865-874 (2005).

Unlike these particle-based agents, the Rapid Deployment Hemostat™ appears to exert its haemostatic effect through red blood cell aggregation, platelet activation, clotting cascade activation and local vasoconstriction. The Rapid Deployment Hemostat™ is an algae-derived dressing composed of poly-N-acetyl-glucosamine. While the original dressing design was effective in reducing minor bleeding, it was necessary to add gauze backing in order to reduce blood loss in swine models of aortic and liver injury. See McManus et al., Business Briefing: Emergency Medical Review 2005, at 78.

Another poly-N-acetyl-glucosamine-derived dressing is the HemCon™ Chitosan Bandage, which is a freeze-dried chitosan dressing purportedly designed to optimize the mucoadhesive surface density and structural integrity of the chitosan at the site of the wound. The HemCon™ Chitosan Bandage apparently exerts its haemostatic effects primarily through adhesion to the wound, although there is evidence suggesting it may also enhance platelet function and incorporate red blood cells into the clot it forms on the wound. This bandage has shown improved hemostasis and reduced blood loss in several animal models of arterial hemorrhage, but a marked variability was observed between bandages, including the failure of some due to inadequate adherence to the wound. See McManus et al., Business Briefing: Emergency Medical Review 2005, at 79.

Liquid fibrin sealants, such as Tisseel VH, have been used for years as an operating room adjunct for hemorrhage control. See J. L. Garza et al., J. Trauma 30:512-513 (1990); H. B. Kram et al., J. Trauma 30:97-101(1990); M. G. Ochsner et al., J. Trauma 30:884-887 (1990); T. L. Matthew et al., Ann. Thorac. Surg. 50:40-44 (1990); H. Jakob et al., J. Vasc. Surg., 1:171-180 (1984). The first mention of tissue glue used for hemostasis dates back to 1909. See Current Trends in Surgical Tissue Adhesives: Proceedings of the First International Symposium on Surgical Adhesives, M. J. MacPhee et al., eds. (Lancaster, Pa.: Technomic Publishing Co; 1995). Liquid fibrin sealants are typically composed of fibrinogen and thrombin, but may also contain Factor III/XIIIa, either as a by-product of fibrinogen purification or as an added ingredient (in certain applications, it is therefore not necessary that Factor XIII/Factor IIIa be present in the fibrin sealant because there is sufficient Factor XIII/XIIIa, or other transaminase, endogenously present to induce fibrin formation). As liquids, however, these fibrin sealants have not proved useful for treating traumatic injuries in the field.

Dry fibrinogen-thrombin dressings having a collagen support (e.g. TachoComb™, TachoComb™ H and TachoSil available from Hafslund Nycomed Pharma, Linz, Austria) are also available for operating room use in many European countries. See U. Schiele et al., Clin. Materials 9:169-177 (1992). While these fibrinogen-thrombin dressings do not require the pre-mixing needed by liquid fibrin sealants, their utility for field applications is limited by a requirement for storage at 4° C. and the necessity for pre-wetting with saline solution prior to application to the wound. These dressings are also not effective against high pressure, high volume bleeding. See Sondeen et al., J. Trauma 54:280-285 (2003).

A dry fibrinogen/thrombin dressing for treating wounded tissue is also available from the American Red Cross (ARC). As disclosed in U.S. Pat. No. 6,762,336, this particular dressing is composed of a backing material and a plurality of layers, the outer two of which contain fibrinogen (but no thrombin) while the inner layer contains thrombin and calcium chloride (but no fibrinogen). While this dressing has shown great success in several animal models of hemorrhage, the bandage is fragile, inflexible, and has a tendency to break apart when handled. See McManus et al., Business Briefing: Emergency Medical Review 2005, at 78.; Kheirabadi et al., J. Trauma 59:25-35 (2005). In addition, U.S. Pat. No. 6,762,336 teaches that this bandage should contain 15 mg/cm2 of fibrinogen to successfully pass a porcine arteriotomy test that is less robust than that disclosed in this application (see Example XI). Moreover, although U.S. Pat. No. 6,762,336 discloses that bandages comprising two layers of fibrinogen, each with a concentration of 4 mg/cm2 to 15 mg/cm2 may provide effective control of hemorrhage, it further teaches that “fibrinogen dose is related to quality. The higher dose is associated with more firm and tightly adhered clots. While lower fibrinogen doses are effective for hemorrhage control during the initial 60 minutes, longer term survival will likely depend on clot quality.”

Other fibrinogen/thrombin-based dressings have also been proposed. For example, U.S. Pat. No. 4,683,142 discloses a resorptive sheet material for closing and healing wounds which consists of a glycoprotein matrix, such as collagen, containing coagulation proteins, such as fibrinogen and thrombin. U.S. Pat. No. 5,702,715 discloses a reinforced biological sealant composed of separate layers of fibrinogen and thrombin, at least one of which also contains a reinforcement filler such as PEG, PVP, BSA, mannitol, FICOLL, dextran, myo-inositol or sodium chlorate. U.S. Pat. No. 6,056,970 discloses dressings composed of a bioabsorbable polymer, such as hyaluronic acid or carboxymethylcellulose, and a haemostatic composition composed of powdered thrombin and/or powdered fibrinogen. U.S. Pat. No. 7,189,410 discloses a bandage composed of a backing material having thereon: (i) particles of fibrinogen; (ii) particles of thrombin; and (iii) calcium chloride. U.S. Patent Application Publication No. US 2006/0155234 A1 discloses a dressing composed of a backing material and a plurality of fibrinogen layers which have discrete areas of thrombin between them. To date, none of these dressings have been approved for use or are available commercially.

In addition, past efforts to prepare fibrinogen/thrombin solid dressings have always been hampered by the very property that makes them desirable ingredients for treating wounds—their inherent ability to rapidly react under aqueous conditions to form fibrin. The present of Factor XIII results in the mixture results in further conversion of fibrin Ia into cross-linked fibrin II.

The overall coagulation process for a human is shown in FIG. 1. As depicted therein, the conversion of fibrinogen into fibrin I involves the cleavage of two small peptides (A and B) from the alpha (a) and (β) chains of fibrinogen respectively. These small peptides are difficult to detect and monitor directly; the decrease in the molecular weight of the alpha and beta chains, however, resulting from this cleavage can be monitored by gel electrophoresis. Similarly, the conversion of fibrin I to cross-linked fibrin II can be followed by the disappearance on gels of the gamma (γ) chain monomer of fibrinogen (as it is converted into γ-γ dimers by the action of Factor XIII upon the γ chain monomers).

To avoid premature reaction, previous attempts to manufacture fibrinogen/thrombin solid dressings have emphasized the separation of the fibrinogen and thrombin components as much as possible in order to prevent them from forming too much fibrin prior to use of the dressing. For example, the fibrinogen-thrombin dressings have a collagen support (e.g. TachoComb™, TachoComb™ H and TachoSil) available from Hafslund Nycomed Pharma are prepared by suspending particles of fibrinogen and thrombin in a non-aqueous liquid and then spraying the suspension onto the collagen base. The use of a non-aqueous environment, as opposed to an aqueous one, is intended to prevent excessive interaction between the fibrinogen and thrombin.

Alternatives to this process have been proposed, each similarly designed to maintain the fibrinogen and thrombin as separately as possible. For example, the fibrinogen/thrombin solid dressing disclosed in U.S. Pat. No. 7,189,410 was prepared by mixing powdered fibrinogen and powdered thrombin in the absence of any solvent and then applying the dry powder mixture to the adhesive side of a backing material. The fibrinogen/thrombin solid dressings disclosed in U.S. Pat. No. 6,762,336 and U.S. Patent Application No. US 2006/0155234 A1 contain separate and discrete layers of fibrinogen or thrombin, each substantially free of the other. These approaches, however, have not been completely successful.

In order to function properly, a fibrinogen/thrombin-based solid dressing must meet several criteria. To begin with, the fibrinogen and thrombin must be able to successfully interact to form a clot and the more this clot adheres to the wound, the better the dressing performs. Grossly, the dressing must have a high degree of integrity, as the loss of active ingredients due to cracking, flaking and the like will ultimately result in decreased performance and meet with poor user acceptance. There have been reports that known fibrinogen/thrombin solid dressings are deficient in one or more of these characteristics.

Furthermore, the dressing must be homogenous, as all areas of the dressing must function equally well in order to assure its successful use. The dressing must also hydrate rapidly and without significant or special efforts. Relatively flat dressings are generally preferred, with curling or irregular, non-planar structures to be avoided if possible (these tend to interfere with effective application and, in some instances, may result in poor performance). Flexibility is another characteristic that is greatly preferred, both to improve performance and to increase the number of wound geometrics and locations that can be treated effectively. Although known fibrinogen/thrombin solid dressings may be flexible when hydrated, they do not possess sufficient moisture content prior to hydration to be flexible. See, e.g., Sondeen et al., J. Trauma 54:280-285 (2003)); Holcomb et al., J. Trauma, 55 518-526; McManus & Wedmore, Emergency Medicine Review, pp76-′79, 2005.

The amount of fibrin present in the dressing prior to use, particularly insoluble, cross-linked fibrin II, must be relatively small. This latter characteristic is important for several reasons. First, the presence of insoluble fibrin during manufacture normally results in poor quality dressings, which can exhibit decreased integrity, lack of homogeneity and difficult/slow hydration. These consequences can usually be detected visually by one of skill in the art.

For example, the presence of pre-formed fibrin in a fibrinogen/thrombin-based solid dressing can be detected visually by deviations from a homogenous surface appearance. In particular, a rough or lumpy appearance frequently signals that there are significant masses of fibrin that have formed during manufacture and will likely impede future performance. Solid, smooth & glossy “sheets’ on the surface of solid dressings are also signs of fibrin that will tend to slow (or even block) hydration during use. Excessive curling up of a solid dressing is another sign that a significant amount of fibrin has formed during manufacture. Upon addition of water or an aqueous solution, dressings with excessive fibrin content are slow to hydrate and often require forceful application of the liquid, sometimes with mechanical penetration of the surface, in order to initiate hydration. Moreover, once hydrated, dressings with a significant amount of pre-formed fibrin usually have a mottled and distinctly non-homogenous appearance.

The amount of pre-formed fibrin can also be assessed by various biochemical assays, such as the method described in U.S. Patent Application Publication No. US 2006/0155234 A1. According to this assay, the conversion of the fibrinogen γ chains to cross-linked γ-γ dimers is used as an indication of the presence of fibrin (the proportion of γ chain that is converted to γ-γ dimer being a measure of the amount of fibrin produced).

Other assays could assess changes in the other component chains of fibrinogen, such as the conversion of the Aα chain into free α chain and fibrinopeptide. A or the conversion of the Bβ chain into free β chain and fibrinopeptide B. These changes can be monitored by gel electrophoresis in a similar manner to the γ to γ-γ conversion described in U.S. Patent Application Publication No. US 2006/0155234 A1. Interestingly, in U.S. Patent Application Publication No. US 2006/0155234 A1, relatively high levels of γ-γ dimerization (up to 10%) were reported, indicating that these dressings included substantial amounts of fibrin prior to use. This observation may account for the delamination and/or cracking observed in some of these dressings.

For a properly functioning fibrinogen/thrombin-based solid dressing, hydration should normally be completed within a few seconds and require nothing more than applying water (or some aqueous solution) onto the dressing. This solution could be blood or another bodily fluid from an injury site that the dressing is applied to, or it may be from some external source, such as a saline or other physiologically acceptable aqueous liquid applied to the dressing while it is on the wound to be treated. Longer hydration times, i.e. generally greater than 5 seconds, will impede the dressing's performance as portions of the dressing may be lost or shed into the fluids which will continue to freely flow prior to formation of sufficient cross-linked fibrin. Given the potentially fatal consequences of continued bleeding, any delay in dressing hydration during use is highly undesirable. In addition, the performance of dressings with excessive fibrin content are usually poor, as reflected by decreased scores in the EVPA and Adherence assays described herein, as well as during in vivo tests and clinical use.

Accordingly, there remains a need in the art for a solid dressing that can be used to treat wounded tissue, particularly wounded tissue resulting from traumatic injury in the field.

This invention relates to processes for the mixing of fibrinogen with thrombin under conditions that limit their interaction to form fibrin, until that interaction is desired. An application for such a process would be in the manufacturing of a fibrin sealant-based haemostatic dressing where the fibrinogen and thrombin mixture would not generate significant levels of fibrin until it is desired that they do so, such as when the dressing is applied to wounded tissue. Such products could have differing fibrinogen/thrombin ratios, and differing ratios within a specific product, in order to maximize the efficacy of the product while minimizing its expense.

The invention also relates to compositions of mixtures containing fibrinogen and thrombin which have levels of fibrin that are sufficiently low so as to permit adequate conversion of fibrinogen to fibrin during application to the patient to ensure the effective use of the product.

The invention also relates to methods of treating a patient in need of therapy with a composition or product made by the processes described above.

Currently, single donor fibrin sealants are widely used clinically, not only for hemorrhage control but in various surgical situations. (W. D. Spotnitz, Thromb. Haemost. 74:482-485 (1995); R. Lerner et al., J. Surg. Res. 48:165-181 (1990)). Even more extensive use is limited by the strict requirements for temperature control, availability of thawed blood components, and the need for mixing of components. Additional problems with the standard fibrin sealants stem from the transfusion risk of human cryoprecipitate (E. M. Soland et al., JAMA 274:1368-1373 (1995)), the low and variable amounts of fibrinogen in the cryoprecipitate (10-30 mg) (P. M. Ness et al., JAMA 241:1690-1691 (1979)), hypotensive responses to bovine thrombin (R. Berguer et al., J. Trauma 31:408-411 (1991)) and antibody responses to bovine thrombin (S. J. Rapaport et al., Am. J. Clin. Pathol. 97:84-91 (1992)).

The American Red Cross and others have developed plasma protein purification methods that seem to eliminate the hepatitis risk. R. F. Reiss et al., Trans. Med. Rev. 10:85-92 (1996). These products are presently being considered for approval by the Food and Drug Administration.

Fibrinogen, thrombin and Factor XIII are 3 proteins that are part of the blood clotting cascade of animals. Briefly, when prothrombin is ‘activated’ to form thrombin, this cleaves off segments from fibrinogen which then self-polymerizes into a soluble fibrin polymer. Thrombin also activates Factor XIII to Factor XIIIa which then catalysis the cross-linking of the fibrin polymer to form a meshwork or net-like, insoluble structure. If the surrounding environment contains injured tissue, Factor XIIIa also crosslinks the fibrin to the tissue, sealing off injured tissue and blood vessels. Many products have been made using some or all of these proteins alone or in combinations with other ingredients (Tissue Sealants Available Today. MacPhee, M & Wilmer, K. in Tissue Glues In Cosmetic Surgery. Renato Saltz & Dean M. Toriumi, Eds. Quality Medical Publishing, Inc. 2004.), however all of these products rely upon maintaining a degree of separation between the reactants prior to application to the patient in order to prevent fibrin formation from proceeding prior to application to the patient's injured tissues. This is required because once fibrin has been fully crosslinked, it will no longer be bound to tissue by the action of Factor XIIIa, and the resulting product will have limited utility for hemostasis or the majority of additional desirable properties of fibrin sealants.

This constraint has limited the scope of inventions and applications for this material, as well as placing manufacturing constraints upon products that result in complex and/or expensive production processes, and producing products with sub-optimal characteristics.

Examples of these include the fibrin sealant-based wound dressings made by NycoMed and the American Red Cross (see U.S. Pat. Nos. 5,942,278; 6,762,336 and PCT Application PCT/US2003/028100).

For example, the manufacture of a haemostatic bandage (U.S. Pat. No. 6,762,336) involves a multi-step manufacturing process that places fibrinogen and thrombin into separate layers. The purpose of the separate layers was to minimize the fibrinogen/thrombin interaction so fibrin would not be formed during the manufacturing process. The resulting product, although effective, is subject to delamination during shipping and handling. Indeed, this deficiency led to the imposition of an even more complex structure and attendant manufacturing process involving an interrupted layer of thrombin (US Patent Application 20060155234: Haemostatic dressing. MacPhee et al, Jul. 13, 2006). If one could mix fibrinogen and thrombin together in a single step, under conditions that minimize fibrin formation, then a simpler manufacturing process that would produce a more robust product, at a reduced manufacturing cost and complexity, with an increased throughput would be possible.

However, as explained above, fibrin, the usual product of the mixing of fibrinogen and thrombin, is itself only weakly haemostatic (D. B. Kendrick, Blood Program in WW II Washington. D.C.: Office of the Surgeon General, Department of Army; 1989. 363-368 & Tissue Sealants Available Today. MacPhee, M & Wilmer, K. in Tissue Glues In Cosmetic Surgery. Renato Saltz & Dean M. Toriumi, Eds. Quality Medical Publishing, Inc. 2004) as compared to the effectiveness of a mixture of fibrinogen, thrombin and factor XIII that does not polymerize before contact with the wound to be treated but rather polymerizes in situ after placed in contact with the wound. This is the reason that fibrin sealant products are manufactured so as to maintain effective separation between at least the thrombin component and the fibrinogen/factor XIII component(s). This is generally accomplished by either drying and packaging the components separately as with conventional fibrin sealants, or by constructing a structure in which the components are layered upon each other under conditions that prevent their interaction (See U.S. Pat. No. 6,762,336).

The extent to which thrombin has interacted with fibrinogen and factor XIII can be determined by measuring the extent to which the native fibrinogen has undergone conversion to fibrin. One of the direct effects of thrombin upon fibrinogen is to remove several small portions of two of the three protein chains comprising the intact fibrinogen molecule. The result is the release of the peptides referred to as fibrinopeptides a and b. This loss can be determined by several methods known in the art, including the change in the molecular weight of the A a and B b chains as they are converted into A & B by the release of the a and b fibrinopeptides respectively. Furthermore thrombin acts upon Factor XIII by removing from it a small peptide, converting the inactive Factor XIII into the active form, known as Factor XIIIa. The effect of Factor XIIIa upon fibrinogen is to form covalent bonds between adjacent fibrinogen γ chains. This converts single γ chain monomers into γ γ dimers. The resulting loss of the γ monomer and appearance of γ γ dimers can also be measured by several techniques known to those skilled in the art, with a simple example being the use of electrophoresis to measure the apparent molecular weights of the components of fibrinogen-based compositions.

Thus the extent to which the three components, fibrinogen, thrombin and factor XIII have interacted can be quantified by several methods. Generally, these involve measuring the proportion of conversion of the fibrinogen chains from their native form to their state within fibrin. This can be accomplished by first measuring the amount of native and/or fibrin form in a composition, then repeating the same measurement(s) on the same composition after first placing the composition for a suitable time into an environment in which the reaction of the components will be completed. Dividing the amount of material in the fibrin form in the initial composition by the amount formed by the complete reaction of the composition determines the proportion of the initial composition that had reacted to form fibrin and thus will not contribute significantly to the haemostatic action of the composition. This can be accomplished for example, by measuring the amount of A a that converts to A, the amount of B b that is converted into B or the amount of γ γ dimer formation.

This requirement to prevent the interaction between fibrinogen, thrombin and factor XIII has limited the nature, structures and manufacturing process of fibrinogen-thrombin based products. Furthermore it has led to complex structures and production process Thus new or improved products could be made if it were possible to manufacture a product by mixing fibrinogen±Factor XIII and thrombin together in a manner that limits fibrin formation.

This patent describes monolithic compositions of fibrinogen±factor XIII and thrombin that remain active and capable of reacting with each other to subsequently form fibrin. These compositions are described in liquid, frozen and solid states. Additionally, manufacturing processes by which these components are combined under conditions that minimize fibrin formation. The resulting compositions and their uses are also described.

### SUMMARY OF THE INVENTION

It is therefore an object of the present invention to provide a solid dressing that can treat wounded mammalian tissue, particularly wounded tissue resulting from a traumatic injury. It is further an object of the present invention to provide a method of treating wounded mammalian tissue, particularly human tissue. Other objects, features and advantages of the present invention will be set forth in the detailed description of preferred embodiments that follows, and will in part be apparent from that description and/or may be learned by practice of the present invention. These objects and advantages will be realized and attained by the compositions and methods described in this specification and particularly pointed out in the claims that follow.

It is therefore a further object of the present invention to produce compositions comprising fibrinogen±Factor XIII, thrombin and fibrin in suitable relative proportions and absolute quantities that may be used to make an effective wound dressing, such as a monolithic dressing or bandage. It is also an object of the invention to treat patients in need thereof using compositions comprising fibrinogen±Factor XIII, thrombin and fibrin in suitable relative proportions and absolute quantities. Other objects, features and advantages of the present invention will be set forth in the detailed description of preferred embodiments and appended claims that follow, and in part will be apparent from that description or may be learned by the practice of the invention. These objects and advantage of the invention will be attained by the compositions, processes and methods particularly pointed out in the written description and claims hereof.

In accordance with these and other objects, a first embodiment of the present invention is direct to a solid dressing for treating wounded tissue in a mammal comprising at least one haemostatic layer consisting essentially of a fibrinogen component and a fibrinogen activator, wherein the haemostatic layer(s) is cast or formed from a single aqueous solutions containing the fibrinogen component and the fibrinogen activator.

In accordance with these and other objects, a first embodiment of the present invention is direct to a solid dressing for treating wounded tissue in a mammal comprising at least one haemostatic layer consisting essentially of fibrinogen and a fibrinogen activator, wherein the fibrinogen is present in an amount between about 3.0 mg/cm² of the surface area of the wound facing side of the dressing and 13.0 mg/cm² of the surface area of the wound facing side of the dressing.

Another embodiment is directed to a solid dressing for treating wounded tissue in a mammal comprising at least one haemostatic layer consisting essentially of a fibrinogen component and a fibrinogen activator, wherein the haemostatic layer(s) is cast or formed as a single piece.

Another embodiment is directed to a method of treating wounded tissue using a solid dressing comprising at least one haemostatic layer consisting essentially of a fibrinogen component and a fibrinogen activator, wherein the haemostatic layer(s) is cast or formed from a single aqueous solution containing the fibrinogen component and the fibrinogen activator.

Another embodiment is directed to a method of treating wounded tissue using a solid dressing comprising at least one haemostatic layer consisting essentially of fibrinogen component and a fibrinogen activator, wherein the haemostatic layer(s) is cast or formed as a single piece.

Another embodiment is directed to a composition consisting essentially of a mixture of fibrinogen component, a fibrinogen activator and water, wherein the composition is frozen and is stable at reduced temperature for at least 24 hours.

Another embodiment is directed to a method of treating wounded tissue using a solid dressing comprising at least one haemostatic layer consisting essentially of fibrinogen and a fibrinogen activator, wherein the fibrinogen is present in an amount between about 11.0 mg/cm2 of the surface area of the wound facing side of the dressing and 13.0 mg/cm2 of the surface area of the wound facing side of the dressing.

In accordance with these and other objects, a first embodiment of the present invention is direct to a solid dressing for treating wounded tissue in a mammal comprising at least one haemostatic layer consisting essentially of a fibrinogen component and thrombin, wherein the thrombin is present in an amount between about 0.250 Units/mg of fibrinogen component and 0.062 Units/mg of fibrinogen component.

Another embodiment is directed to a method of treating wounded tissue using a solid dressing comprising at least one haemostatic layer consisting essentially of a fibrinogen component and thrombin, wherein the thrombin is present in an amount between about 0.250 Units/mg of fibrinogen component and 0.062 Units/mg of fibrinogen component.

Other embodiments are directed to similar solid dressings wherein the amount of thrombin is between 0.125 Units/mg of fibrinogen component and 0.080 Units/mg of fibrinogen component., and the use of the same for treating wounded tissue.

It is to be understood that the foregoing general description and the following detailed description of preferred embodiments are exemplary and explanatory only and are intended to provide further explanation, but not limitation, of the invention as claimed herein.

### BRIEF DESCRIPTION OF DRAWINGS

FIG. 1 is an overview of the human clotting cascade as provided by ERL's website (www.enzymeresearch.co.uk/coag.htm).

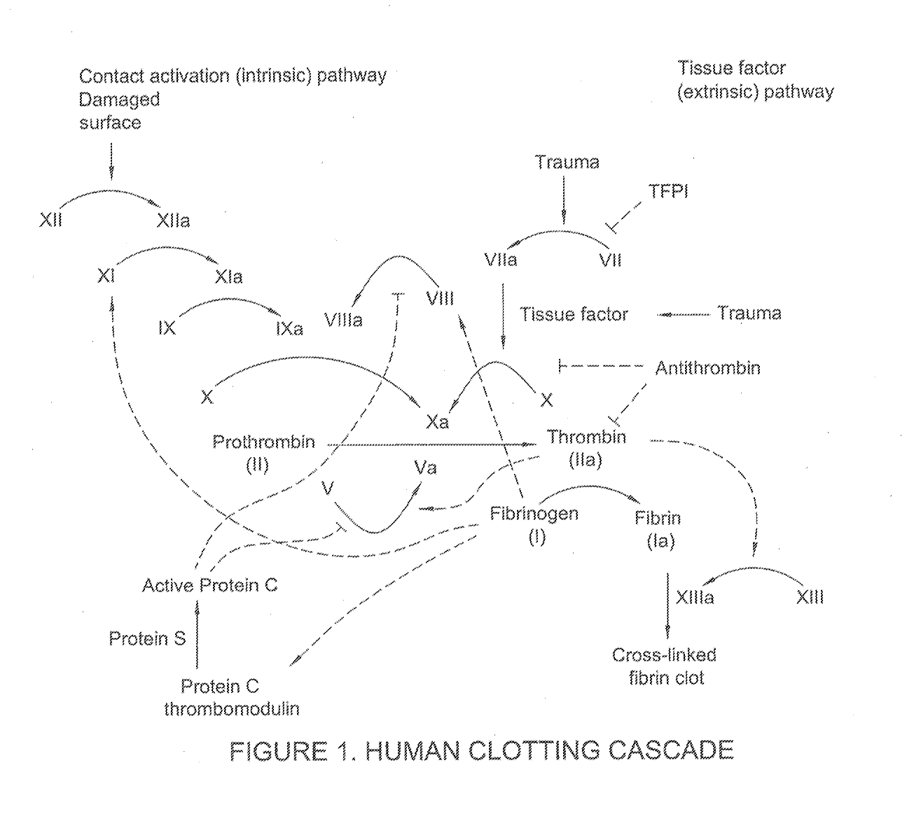

FIG. 2 is a diagram of the set-up for the ex vivo porcine arteriotomoy assay described herein.

FIGS. 3A-3C are graphs showing the results achieved in Example 1.

FIG. 4A and FIG. 4B are graphs depicting the results of the EVPA and Adherence Assays for the dressings made in Examples 6-12.

FIGS. 5A and 5B are graphs showing the performance characteristics of frozen compositions stored at −80° C. as shown in Example 13.

### DETAILED DESCRIPTION OF PREFERRED EMBODIMENTS

Unless defined otherwise, all technical and scientific terms used herein have the same meaning as is commonly understood by one of skill in the art to which this invention belongs. All patents and publications mentioned herein are incorporated by reference.

As used herein, use of a singular article such as “a,” “an,” and “the” is not intended to excluded pluralities of the article's object unless the context clearly and unambiguously dictates otherwise.

“Dressing” as used herein refers to a material applied to a wound with the intension of treating the wound in such a manner as to limit, eliminate or prevent one or more undesirable processes from occurring in or around the application site. This term encompasses related terms such as “bandage”, etc.

“Thrombin” as used herein refers to Coagulation Factor IIa, its pre-cursors and derivatives. Thrombin may be used to convert fibrinogen to Fibrin I. It may also be used to convert Blood Coagulation Factor XIII to Factor XIIIa, which in turn is able to convert Fibrin I to insoluble, cross linked Fibrin II. If a composition contains all three of Fibrinogen, Thrombin and Factor XIII, then the action of Thrombin may be in effect to convert Fibrinogen to Fibrin. As used herein, unless explicitly stated otherwise, the use of “Thrombin” in a process or composition of the invention also encompasses the use of any other substance that is known to those skilled in the art to cause the conversion of Fibrinogen to one or more forms of Fibrin. Illustrative examples of “Thrombin-equivalents” include, but are not limited to, Thrombin-like enzymes found in snake venoms, such as ancrod, batroxobin, calobin and flavoxobin. The selection of Thrombin and/or a Thrombin-equivalent for use in a particular process or composition of the invention may vary and the particular choice required may be made empirically by one skilled in the art.

“Mold” as used herein refers to a structure or container that either restrains the movement of a composition, or defines its extent in one or more dimensions. A mold may be used merely to form a composition into a desired shape. Alternately, a mold may serve both that function and also one or more additional functions, such as providing a component of a system designed to isolate the composition from the surrounding environment, or to protect it from external alteration by heat or physical shock. Accordingly, a mold may be used temporarily for only a portion of the process required to form a composition, which may then be removed from the mold and the mold discarded or re-used. Alternately, a mold may serve the initial function of giving form to the composition, and be employed subsequently as a container or a portion of a container for the product. The molds may have various connectors and ports that allow the introduction of various compositions into the mold, and/or the escape of the interior atmosphere during filling and/or lyophilization or other drying step. The molds may be fabricated into a single piece, or have one or more movable or removable components to facilitate manufacture or storage.

“Filling” as used herein refers to adding one or more components of a composition to a container or mold. Unless otherwise specified, two or more of the components may be mixed prior to addition to the container or mold. Alternately, two or more of the components may be added sequentially or simultaneously to the container or mold. The resulting mixture may be homogenous or incompletely mixed according to the desired function. The volume used to fill a container may be any useful quantity relative to the volume of the container or mold. When the container or mold has at least one dimension that is longer than another, the filling may be performed with the container or mold in any suitable orientation. For example, if the long axis of the container is oriented horizontally, then the filling of said container while in this orientation is said to be “Horizontal”. Conversely, when the filling takes place while the long axis is oriented vertically the filling is said to be ‘Vertical”. The filling can be carried out with a substantial opening to the surrounding atmosphere exists in the container or mold, such that the area of the opening(s) is/are substantially greater than the area of the opening(s) used to fill the container. This is referred to as “Open Filling” or “Open Mold Filling”. In contrast, when there is no opening of the container that connects unimpeded to the surrounding atmosphere the filling of the mold is said to be a “Closed Filling” or “Closed Mold” filling. When the composition(s) to be filled into said closed filling system is introduced under pressure this is referred to as “Injection Mold Filling” or “Injection Molding”. The container or molds may be at or above ambient temperatures during filling, or below ambient temperature so as to facilitate a rapid freezing of the filled components. Filling may be carried out at such a rate as to permit the effective mixing of the components prior to their freezing into a monolithic mass.

“Haemostatic agent” as used herein is a composition or product that when applied to a patient with at least one site of active bleeding results in a reduction in the rate of blood loss.

“Patient” as used herein refers to human or animal individuals in need of medical care and/or treatment.

“Wound” as used herein refers to any damage to any tissue of a patient which results in the loss of blood from the circulatory system and/or any other fluid from the patient's body. The tissue may be an internal tissue, such as an organ or blood vessel, or an external tissue, such as the skin. The loss of blood may be internal, such as from a ruptured organ, or external, such as from a laceration. A wound may be in a soft tissue, such as an organ, or in hard tissue, such as bone. The damage may have been caused by any agent or source, including traumatic injury, infection or surgical intervention.

“Resorbable material” as used herein refers to a material that is broken down spontaneously and/or by the mammalian body into components which are consumed or eliminated in such a manner as not to interfere significantly with wound healing and/or tissue regeneration, and without causing any significant metabolic disturbance.

“Stability” as used herein refers to the retention of those characteristics of a material that determine activity and/or function.

“Suitable” as used herein is intended to mean that a material does not adversely affect the stability of the dressings or any component thereof.

“Binding agent” as used herein refers to a compound or mixture of compounds that improves the adherence and/or cohesion of the components of the haemostatic layer(s) of the dressings.

“Solubilizing agent” as used herein refers to a compound or mixture of compounds that improves the dissolution of a protein or proteins in aqueous solvent.

“Filler” as used herein refers to a compound or mixture of compounds that provide bulk and/or porosity to the haemostatic layer(s) of a dressing.

“Release agent” as used herein refers to a compound or mixture of compounds that facilitates removal of a dressing from a manufacturing mold.

“Foaming agent” as used herein refers to a compound or mixture of compounds that produces gas when hydrated under suitable conditions.

“Solid” as used herein is intended to mean that the dressing will not substantially change in shape or form when placed on a rigid surface, wound-facing side down, and then left to stand at room temperature for 24 hours.

“Monolithic” as used herein refers to a composition that is formed so as to have a single layer with all ingredients within that layer. A backing material may be added to the surface of, or within such a composition without changing its designation as ‘monolithic’.

“Dried” refers to a composition that has had enough of the available water removed from it such that the composition is substantially solid, but not frozen. Suitable methods for drying materials are known and/or can be determined by those skilled in the art, and include; evaporation, sublimation, heating, lyophilizing, spinning, electrospinning (see U.S. Pat. No. 1,975,504, J. Electrostatics 35, 151 (1995) and Polymer, 40, (1999)), concentration, spray drying, liquid crystallization, pressing, crystallization and combinations of two or more such techniques.

“Frozen” as used herein is intended to mean that the composition will not substantially change in shape or form when placed on a rigid surface, wound-facing side down, and then left to stand at −40° C. for 24 hours, but will substantially change in shape or form when placed on a rigid surface, wound-facing side down, and then left at room temperature for 24 hours. Thus, in the context of the present invention, a “solid” dressing is not “frozen” and a “frozen” composition is not “solid”.

“Lyophilized” as used herein refers to refers to material that has had some of its available water removed by freezing the material and then reducing the pressure surrounding it. This process is synonymous with “Freeze-drying”. The reduction in the available water may be sufficient that the material may exist as a solid at temperatures at which it would have been a liquid prior to lyophilization.

“Cooling” as used herein refers to the process of lowering the temperature of an object or composition. There are three fundamental processes by which cooling may take place. These are referred to as Convective, Conductive and Radiative cooling (Introduction to the Principals of Heat Transfer, Website available at: http://www.efunda.com/formulae/heat.transfer/home/overview.cfm Jul. 19, 2006). In practice it is difficult to cool an object by only one of these mechanisms, however cooling processes can be devised in which one or two of these mechanisms predominate. An example of this is the industrial process of blast cooling or blast freezing. In this process, a large volume of cooled air or other gas is forced past the object(s) to be cooled. The majority of the heat energy removed from the object is transferred to the moving gas and removed via convection. This type of convective cooling is referred to as “Forced Convection”. This form of cooling is often augmented by the introduction into the cooling gas of a cryogenic liquid, such as liquid nitrogen, to produce a very low temperature cooling gas and reduce the cooling or freezing time. Conductive cooling can predominate when a cooled block of material is placed in contact with the object to be cooled. Radiative cooling can dominate when an object to be cooled is placed in close proximity, but not in contact, with a cooled object.

A first preferred embodiment of the present invention is directed to a solid dressing for treating wounded tissue in a patient which comprises a haemostatic layer consisting of a fibrinogen component and a fibrinogen activator, wherein the haemostatic layer(s) is cast or formed from a single aqueous solution containing the fibrinogen component and the fibrinogen activator.

A second preferred embodiment of the present invention is directed to a solid dressing for treating wounded tissue in a patient which comprises a haemostatic layer consisting of fibrinogen component and thrombin, wherein the thrombin is present in an amount between 0.250 Units/mg of fibrinogen component and 0.062 Units/mg of fibringogen component.

A third preferred embodiment of the present invention is directed to a solid dressing for treating wounded tissue in a patient which comprises a haemostatic layer consisting of fibrinogen and a fibrinogen activator, wherein the fibrinogen is present in an amount between 3.0 mg/cm² of the surface area of the wound facing side of the dressing and 13.0 mg/cm² of the surface area of the wound facing side of the dressing, all values being ±0.09 mg/cm².

Another embodiment of the present invention is directed to a solid dressing for treating wounded tissue in a patient which comprises a haemostatic layer consisting of a fibrinogen component and a fibrinogen activator, wherein the haemostatic layer(s) is cast or formed as single piece.

As used herein, “consisting essentially of” is intended to mean that the fibrinogen and the fibrinogen activator are the only necessary and essential ingredients of the haemostatic layer(s) of the solid dressing when it is used as intended to treat wounded tissue. Accordingly, the haemostatic layer may contain other ingredients in addition to the fibrinogen component and the fibrinogen activator as desired for a particular application, but these other ingredients are not required for the solid dressing to function as intended under normal conditions, i.e. these other ingredients are not necessary for the fibrinogen component and fibrinogen activator to react and form enough fibrin to reduce the flow of blood and/or fluid from normal wounded tissue when that dressing is applied to that tissue under the intended conditions of use. If, however, the conditions of use in a particular situation are not normal, for example the patient is a hemophiliac suffering from Factor XIII deficiency, then the appropriate additional components, such as Factor III/XIIIa or some other transaminase, may be added to the haemostatic layer(s) without deviating from the spirit of the present invention. Similarly, the solid dressing of the present invention may contain one or more of these haemostatic layers as well as one or more other layers, such as one or more support layers (e.g. a backing material or an internal support material) and release layers.

Other preferred embodiments are directed to similar solid dressings wherein the amount of thrombin is between 0.125 Units/mg of fibrinogen component and 0.080 Units/mg of fibrinogen component. Still other preferred embodiments of the present invention are directed to similar solid dressings wherein the amount of thrombin is (all values being ±0.0009): 0.250 Units/mg of fibrinogen component; 0.125 Units/mg of fibrinogen component; 0.100 Units/mg of fibrinogen component; 0.080 Units/mg of fibrinogen component; 0.062 Units/mg of fibrinogen component; 0.050 Units/mg of fibrinogen component; and 0.025 Units/mg of fibrinogen component.

Another preferred embodiment of the present invention is directed to a method for treating wounded tissue in a mammal, comprising placing a solid dressing of the present invention to wounded tissue and applying sufficient pressure to the dressing for a sufficient time for enough fibrin to form to reduce the loss of blood and/or other fluid from the wound.

Other preferred embodiments of the present invention are directed to methods for treating wounded tissue in a mammal, comprising placing a solid dressing of the present invention to wounded tissue and applying sufficient pressure to the dressing for a sufficient time for enough fibrin to form to reduce the loss of blood and/or other fluid from the wound.

Other preferred embodiments of the present invention include similar solid dressings wherein the fibrinogen is present in an amount between 11.0 mg/cm2 of the surface area of the wound facing side of the dressing and 13.0 mg/cm2 of the surface area of the wound facing side of the dressing, all values being ±0.09 mg/cm2. Other preferred embodiments include similar solid dressings wherein the fibrinogen is present in an amount between 3.0 mg/cm2 and 9.0 mg/cm2 Still other preferred embodiments are directed to similar solid dressings wherein the amount of fibrinogen is: 3.0 mg/cm2 of the surface area of the wound facing side of the dressing; 5.0 mg/cm2; 7.0 mg/cm2; 9.0 mg/cm2; 11.0 mg/cm2; or 13.0 mg/cm2 (all values being ±0.09 mg/cm2).

Still other preferred embodiments are directed to compositions consisting essentially of a mixture of a fibrinogen component, a fibrinogen activator and water, wherein these compositions are frozen and are stable at reduced temperature for at least 24 hours. Such compositions are particularly useful for preparing the haemostatic layer(s) of the inventive solid dressings.

According to certain embodiments of the present invention, the haemostatic layer(s) of the solid dressing is formed or cast as a single piece. According to certain other embodiments of the present invention, the haemostatic layer is made or formed into or from a single source, e.g. an aqueous solution containing a mixture of the fibrinogen and the fibrinogen activator. With each of these embodiments of the present invention, the haemostatic layer(s) is preferably substantially homogeneous throughout.

According to other preferred embodiments, the haemostatic layer(s) of the solid dressing are composed of a plurality of particles, each of which consists essentially of fibrinogen component and thrombin. According to such embodiments, the haemostatic layer may also contain a binding agent to facilitate or improve the adherence of the particles to one another and/or to any support layer(s). Illustrative examples of suitable binding agents include, but are not limited to, sucrose, mannitol, sorbitol, gelatin, hyaluron and its derivatives, such as hyaluronic acid, povidone, starch, chitosan and its derivatives (e.g., NOCC-Chitosan), and cellulose derivatives, such as carboxymethylcellulose, as well as mixtures of two or more thereof.

According to other preferred embodiments, the haemostatic layer(s) of the solid dressing may also contain a binding agent to facilitate or improve the adherence of the layer(s) to one another and/or to any support layer(s). Illustrative examples of suitable binding agents include, but are not limited to, sucrose, mannitol, sorbitol, gelatin, hyaluron and its derivatives, such as hyaluronic acid, maltose, povidone, starch, chitosan and its derivatives, and cellulose derivatives, such as carboxymethylcellulose, as well as mixtures of two or more thereof.

According to other preferred embodiments, the haemostatic layer(s) of the solid dressing are composed of a plurality of particles, each of which consists essentially of fibrinogen and a fibrinogen activator. According to such embodiments, the haemostatic layer may also contain a binding agent to facilitate or improve the adherence of the particles to one another and/or to any support layer(s). Illustrative examples of suitable binding agents include, but are not limited to, sucrose, mannitol, sorbitol, gelatin, hyaluron and its derivatives, such as hyaluronic acid, maltose, povidone, starch, chitosan and its derivatives, and cellulose derivatives, such as carboxymethylcellulose, as well as mixtures of two or more thereof.

The haemostatic layer(s) of the solid dressing may also optionally contain one or more suitable fillers, such as sucrose, lactose, maltose, silk, fibrin, collagen, albumin, polysorbate (Tween™), chitin, chitosan and its derivatives, (e.g. NOCC-chitosan), alginic acid and salts thereof, cellulose and derivatives thereof, proteoglycans, hyaluron and its derivatives, such as hyaluronic acid, glycolic acid polymers, lactic acid polymers, glycolic acid/lactic acid co-polymers, and mixtures of two or more thereof.

The haemostatic layer of the solid dressing may also optionally contain one or more suitable solubilizing agents, such as sucrose, dextrose, mannose, trehalose, mannitol, sorbitol, albumin, hyaluron and its derivatives, such as hyaluronic acid, sorbate, polysorbate (Tween™), sorbitan (SPAN™) and mixtures of two or more thereof.

The haemostatic layer of the solid dressing may also optionally contain one or more suitable foaming agents, such as a mixture of a physiologically acceptable acid (e.g. citric acid or acetic acid) and a physiologically suitable base (e.g. sodium bicarbonate or calcium carbonate). Other suitable foaming agents include, but are not limited to, dry particles containing pressurized gas, such as sugar particles containing carbon dioxide (see, e.g. U.S. Pat. No. 3,012,893) or other physiologically acceptable gases (e.g. Nitrogen or Argon), and pharmacologically acceptable peroxides. Such a foaming agent may be introduced into the aqueous mixture of the fibrinogen component and the fibrinogen activator, or may be introduced into an aqueous solution of the fibrinogen component and/or an aqueous solution of the fibrinogen activator prior to mixing.

The haemostatic layer(s) of the solid dressing may also optionally contain a suitable source of calcium ions, such as calcium chloride, and/or a fibrin cross-linker, such as a transaminase (e.g. Factor III/XIIIa) or glutaraldehyde.

The haemostatic layer of the solid dressing is preferably prepared by mixing aqueous solutions of the fibrinogen and the fibrinogen activator under conditions which minimize the activation of the fibrinogen by the fibrinogen activator. The mixture of aqueous solutions is then subjected to a process such as lyophilization or free-drying to reduce the moisture content to the desired level, i.e. to a level where the dressing is solid and therefore will not substantially change in shape or form upon standing, wound-facing surface down, at room temperature for 24 hours. Similar processes that achieve the same result, such as drying, spray-drying, vacuum drying and vitrification, may also be employed.

As used herein, “moisture content” refers to the amount freely-available water in the dressing. “Freely-available” means the water is not bound to or complexed with one or more of the non-liquid components of the dressing. The moisture content referenced herein refers to levels determined by procedures substantially similar to the FDA-approved, modified Karl Fischer method (Meyer and Boyd, Analytical Chem., 31:215-219, 1959; May et al. J. Biol. Standardization, 10:249-259, 1982; Centers for Biologies Evaluation and Research, FDA, Docket No. 89D-0140, 83-93; 1990) or by near infrared spectroscopy. Suitable moisture content(s) for a particular solid dressing may be determined empirically by one skilled in the art depending upon the intended application) thereof.

For example, in certain embodiments of the present invention, higher moisture contents are associated with more flexible solid dressings. Thus, in solid dressings intended for extremity wounds, it may be preferred to have a moisture content of at least 6% and even more preferably in the range of 6% to 44%.

Similarly, in other embodiments of the present invention, lower moisture contents are associated with more rigid solid dressings. Thus, in solid dressings intended for flat wounds, such as wounds to the abdomen or chest, it may be preferred to have a moisture content of less than 6% and even more preferably in the range of 1% to 6%.

Accordingly, illustrative examples of suitable moisture contents for solid dressings include, but are not limited to, the following (each value being ±0.9%): less than 53%; less than 44%; less than 28%; less than 24%; less than 16%; less than 12%; less than 6%; less than 5%; less than 4%; less than 3%; less than 2.5%; less than 2%; less than 1.4%; between 0 and 12%, non-inclusive; between 0 and 6%; between 0 and 4%; between 0 and 3%; between 0 and 2%; between 0 and 1%; between 1 and 16%; between 1 and 11%; between 1 and 8%; between 1 and 6%; between 1 and 4%; between 1 and 3%; between 1 and 2%; and between 2 and 4%.

The fibrinogen in the haemostatic layer(s) of the solid dressings may be any suitable fibrinogen known and available to those skilled in the art. A specific fibrinogen for a particular application may be selected empirically by one skilled in the art. As used herein, the term “fibrinogen” is intended to include mixtures of fibrinogen and small amounts of Factor XIII/Factor Ma, or some other such transaminase. Such small amounts are generally recognized by those skilled in the art as usually being found in mammalian fibrinogen after it has been purified according to the methods and techniques presently known and available in the art, and typically range from 0.1 to 20 Units/mL.

The fibrinogen component may also be a functional derivative or metabolite of a fibrinogen, such the fibrinogen α, β and/or γ chains, soluble fibrin I or fibrin II, or a mixture of two or more thereof. A specific fibrinogen (or functional derivative or metabolite) for a particular application may be selected empirically by one skilled in the art.

Preferably, the fibrinogen employed as the fibrinogen component of the solid dressing is a purified fibrinogen suitable for introduction into a mammal. Typically, such fibrinogen is a part of a mixture of human plasma proteins which include Factor III/XIIIa and have been purified to an appropriate level and vitally inactivated. A preferred aqueous solution of fibrinogen for preparation of a solid dressing contains around 37.5 mg/mL fibrinogen at a pH of around 7.4±0.1. Suitable fibrinogen for use as the fibrinogen component has been described in the art, e.g. U.S. Pat. No. 5,716,645, and similar materials are commercially available, e.g. from sources such as Sigma-Aldrich, Enzyme Research Laboratories, Haematologic Technologies and Aniara.

Preferably, the fibrinogen component is present in an amount of from about 1.5 to about 13.0 mg (±0.9 mg) of fibrinogen per square centimeter of solid dressing, and more preferably from about 3.0 to about 13.0 mg/cm². Greater or lesser amounts, however, may be employed depending upon the particular application intended for the solid dressing. For example, according to certain embodiments where increased adherence is desired, the fibrinogen component is present in an amount of from about 11.0 to about 13.00 mg (±0.9 mg) of fibrinogen per square centimeter of solid dressing. Likewise, according to certain embodiments which are intended for treating low pressure-containing vessels, lower levels of the fibrinogen component may be employed.

The fibrinogen activator employed in the haemostatic layer(s) of the solid dressing may be any of the substances or mixtures of substances known by those skilled in the art to convert fibrinogen into fibrin. Illustrative examples of suitable fibrinogen activators include, but are not limited to, the following: thrombins, such as human thrombin or bovine thrombin, and prothrombins, such as human prothrombin or prothrombin complex concentrate (a mixture of Factors II, VII, IX and X); snake venoms, such as batroxobin, reptilase (a mixture of batroxobin and Factor Ma), bothrombin, calobin, fibrozyme, and enzymes isolated from the venom of Bothrops jararacussu; and mixtures of any two or more of these. See, e.g., Dascombe et al., Thromb. Haemost. 78:947-51 (1997); Hahn et al., J. Biochem. (Tokyo) 119:835-43 (1996); Fortova et al., J. Chromatogr. S. Biomed. Appl. 694:49-53 (1997); and Andriao-Escarso et al., Toxicon. 35: 1043-52 (1997).

Preferably, the fibrinogen activator is a thrombin. More preferably, the fibrinogen activator is a mammalian thrombin, although bird and/or fish thrombin may also be employed in appropriate circumstances. While any suitable mammalian thrombin may be used in the solid dressing, the thrombin employed in the haemostatic layer is preferably a lyophilized mixture of human plasma proteins which has been sufficiently purified and virally inactivated for the intended use of the solid dressing. Suitable thrombin is available commercially from sources such as Sigma-Aldrich, Enzyme Research Laboratories, Haematologic Technologies and Biomol International. A particularly preferred aqueous solution of thrombin for preparing a solid dressing contains thrombin at a potency of between 10 and 2000±50 International Units/mL, and more preferred at a potency of 25±2.5 International Units/mL. Other constituents may include albumin (generally about 0.1 mg/mL) and glycine (generally about 100 mM±0.1 mM). The pH of this particularly preferred aqueous solution of thrombin is generally in the range of 6.5-7.8, and preferably 7.4±0.1, although a pH in the range of 5.5-8.5 may be acceptable.

In addition to the haemostatic layer(s), the solid dressing may optionally further comprise one or more support layers. As used herein, a “support layer” refers to a material that sustains or improves the structural integrity of the solid dressing and/or the fibrin clot formed when such a dressing is applied to wounded tissue.

According to certain preferred embodiments of the present invention the support layer comprises a backing material on the side of the dressing opposite the side to be applied to wounded tissue. Such a backing material may be affixed with a physiologically-acceptable adhesive or may be self-adhering (e.g. by having a sufficient surface static charge). The backing material may comprise one or more resorbable materials or one or more non-resorbable materials or mixtures thereof. Preferably, the backing material is a single resorbable material.

Any suitable resorbable material known and available to those skilled in the art may be employed in the present invention. For example, the resorbable material may be a proteinaceous substance, such as silk, fibrin, keratin, collagen and/or gelatin. Alternatively, the resorbable material may be a carbohydrate substance, such as alginates, chitin, cellulose, proteoglycans (e.g. poly-N-acetyl glucosamine), hyaluron and its derivatives, such as hyaluronic acid, glycolic acid polymers, lactic acid polymers, or glycolic acid/lactic acid co-polymers. The resorbable material may also comprise a mixture of proteinaceous substances or a mixture of carbohydrate substances or a mixture of both proteinaceous substances and carbohydrate substances. Specific resorbable material(s) may be selected empirically by those skilled in the art depending upon the intended use of the solid dressing.

According to certain preferred embodiments of the present invention, the resorbable material is a carbohydrate substance. Illustrative examples of particularly preferred resorbable materials include, but are not limited to, the materials sold under the trade names VICRYL™ (a glycolic acid/lactic acid copolymer) and DEXON™ (a glycolic acid polymer).

Any suitable non-resorbable material known and available to those skilled in the art may be employed as the backing material. Illustrative examples of suitable non-resorbable materials include, but are not limited to, plastics, silicone polymers, paper and paper products, latex, gauze and the like.

According to other preferred embodiments, the support layer comprises an internal support material. Such an internal support material is preferably fully contained within a haemostatic layer of the solid dressing, although it may be placed between two adjacent haemostatic layers in certain embodiments. As with the backing material, the internal support material may be a resorbable material or a non-resorbable material, or a mixture thereof, such as a mixture of two or more resorbable materials or a mixture of two or more non-resorbable materials or a mixture of resorbable material(s) and non-resorbable material(s).

According to still other preferred embodiments, the support layer may comprise a front support material on the wound-facing side of the dressing, i.e. the side to be applied to wounded tissue. As with the backing material and the internal support material, the front support material may be a resorbable material or a non-resorbable material, or a mixture thereof, such as a mixture of two or more resorbable materials or a mixture of two or more non-resorbable materials or a mixture of resorbable material(s) and non-resorbable material(s).

According to still other preferred embodiments, the solid dressing comprises both a backing material and an internal support material in addition to the haemostatic layer(s), i.e. the solid dressing comprises two support layers in addition to the haemostatic layer(s). According to still other preferred embodiments, the solid dressing comprises both a front support material and an internal support material in addition to the haemostatic layer(s). According to still other preferred embodiments, the solid dressing comprises a backing material, a front support material and an internal support material in addition to the haemostatic layer(s).

According to certain embodiments of the present invention, particularly where the solid dressing is manufactured using a mold, the solid dressings may also optionally further comprise a release layer in addition to the haemostatic layer(s) and support layer(s). As used herein, a “release layer” refers to a layer containing one or more agents (“release agents”) which promote or facilitate removal of the solid dressing from a mold in which it has been manufactured. A preferred such agent is sucrose, but other suitable release agents include gelatin, mannitol, sorbitol, hyaluron and its derivatives, such as hyaluronic acid, mannitol, sorbitol and glucose. Alternatively, such one or more release agents may be contained in the haemostatic layer.

The various layers of the inventive dressings may be affixed to one another by any suitable means known and available to those skilled in the art. For example, a physiologically-acceptable adhesive may be applied to a backing material (when present), and the haemostatic layer(s) subsequently affixed thereto.

In certain embodiments of the present invention, the physiologically-acceptable adhesive has a shear strength and/or structure such that the backing material can be separated from the fibrin clot formed by the haemostatic layer after application of the dressing to wounded tissue. In other embodiments, the physiologically-acceptable adhesive has a shear strength and/or structure such that the backing material cannot be separated from the fibrin clot after application of the bandage to wounded tissue.

Suitable fibrinogens and suitable fibrinogen activators for the haemostatic layer(s) of the solid dressing may be obtained from any appropriate source known and available to those skilled in the art, including, but not limited to, the following: from commercial vendors, such as Sigma-Aldrich and Enzyme Research Laboratories; by extraction and purification from human or mammalian plasma by any of the methods known and available to those skilled in the art; from supernatants or pastes derived from plasma or recombinant tissue culture, viruses, yeast, bacteria, or the like that contain a gene that expresses a human or mammalian plasma protein which has been introduced according to standard recombinant DNA techniques; and/or from the fluids (e.g. blood, milk, lymph, urine or the like) of transgenic mammals (e.g. goats, sheep, cows) that contain a gene which has been introduced according to standard transgenic techniques and that expresses the desired fibrinogen and/or desired fibrinogen activator.

According to certain preferred embodiments of the present invention, the fibrinogen is a mammalian fibrinogen such as bovine fibrinogen, porcine fibrinogen, ovine fibrinogen, equine fibrinogen, caprine fibrinogen, feline fibrinogen, canine fibrinogen, murine fibrinogen or human fibrinogen. According to other embodiments, the fibrinogen is bird fibrinogen or fish fibrinogen. According to still other embodiments, the fibrinogen component is human fibrinogen, human fibrinogen α chain, human fibrinogen β chain, human fibrinogen γ chain, human fibrin I, human fibrin II, or a mixture of two or more thereof. According to any of these embodiments, the fibrinogen may be recombinantly produced fibrinogen or transgenic fibrinogen. As noted above, the fibrinogen may also contain small amounts (e.g. ______-______ % of total protein) of a transaminase, such as Factor XIII/XIIIa.

According to certain preferred embodiments of the present invention, the fibrinogen activator is a mammalian thrombin, such as bovine thrombin, porcine thrombin, ovine thrombin, equine thrombin, caprine thrombin, feline thrombin, canine thrombin, murine thrombin and human thrombin. According to other embodiments, the thrombin is bird thrombin or fish thrombin. According to any of these embodiments, the thrombin may be recombinantly produced thrombin or transgenic thrombin.

As a general proposition, the purity of the fibrinogen and/or the fibrinogen activator for use in the solid dressing will be a purity known to one of ordinary skill in the relevant art to lead to the optimal efficacy and stability of the protein(s). Preferably, the fibrinogen and/or the fibrinogen activator has been subjected to multiple purification steps, such as precipitation, concentration, diafiltration and affinity chromatography (preferably immunoaffinity chromatography), to remove substances which cause fragmentation, activation and/or degradation of the fibrinogen and/or the fibrinogen activator during manufacture, storage and/or use of the solid dressing. Illustrative examples of such substances that are preferably removed by purification include: protein contaminants, such as inter-alpha trypsin inhibitor and pre-alpha trypsin inhibitor; non-protein contaminants, such as lipids; and mixtures of protein and non-protein contaminants, such as lipoproteins.

The amount of the fibrinogen activator employed in the solid dressing is preferably selected to optimize both the efficacy and stability thereof. As such, a suitable concentration for a particular application of the solid dressing may be determined empirically by one skilled in the relevant art. According to certain preferred embodiments of the present invention, when the fibrinogen activator is human thrombin, the amount of human thrombin employed is between 2.50 Units/mg of fibrinogen component and 0.025 Units/mg of the fibrinogen (all values being ±0.0009). Other preferred embodiments are directed to similar solid dressings wherein the amount of thrombin is between 0.250 Units/mg of fibrinogen and 0.062 Units/mg of fibrinogen and solid dressings wherein the amount of thrombin is between 0.125 Units/mg of fibrinogen and 0.080 Units/mg of fibrinogen.

According to certain preferred embodiments of the present invention, when the fibrinogen component is human fibrinogen, the amount of fibrinogen employed is between 1.5 mg and 13.0 mg (each ±0.9 mg) per square centimeter of solid dressing, more preferably between 3.0 mg and 13.0 mg per square centimeter and most preferably between 11.0 mg and 13.0 mg per square centimeter.

During use of the solid dressing, the fibrinogen and the fibrinogen activator are preferably activated at the time the dressing is applied to the wounded tissue by the endogenous fluids of the patient escaping from the hemorrhaging wound. Alternatively, in situations where fluid loss from the wounded tissue is insufficient to provide adequate hydration of the protein layers, the fibrinogen component and/or the thrombin may be activated by a suitable, physiologically-acceptable liquid, optionally containing any necessary co-factors and/or enzymes, prior to or during application of the dressing to the wounded tissue.

In some embodiments of the present invention, the haemostatic layer(s) may also contain one or more supplements, such as growth factors, drugs, polyclonal and monoclonal antibodies and other compounds. Illustrative examples of such supplements include, but are not limited to, the following: fibrinolysis inhibitors, such as aprotonin, tranexamic acid and epsilon-amino-caproic acid; antibiotics, such as tetracycline and ciprofloxacin, amoxicillin, and metronidazole; anticoagulants, such as activated protein C, heparin, prostacyclins, prostaglandins (particularly (PGI₂), leukotrienes, antithrombin III, ADPase, and plasminogen activator; steroids, such as dexamethasone, inhibitors of prostacyclin, prostaglandins, leukotrienes and/or kinins to inhibit inflammation; cardiovascular drugs, such as calcium channel blockers, vasodilators and vasoconstrictors; chemoattractants; local anesthetics such as bupivacaine; and antiproliferative/antitumor drugs such as 5-fluorouracil (5-FU), taxol and/or taxotere; antivirals, such as gangcyclovir, zidovudine, amantidine, vidarabine, ribaravin, trifluridine, acyclovir, dideoxyuridine and antibodies to viral components or gene products; cytokines, such as alpha- or beta- or gamma-Interferon, alpha- or beta-tumor necrosis factor, and interleukins; colony stimulating factors; erythropoietin; antifungals, such as diflucan, ketaconizole and nystatin; antiparasitic gents, such as pentamidine; anti-inflammatory agents, such as alpha-1-anti-trypsin and alpha-1-antichymotrypsin; anesthetics, such as bupivacaine; analgesics; antiseptics; hormones; vitamins and other nutritional supplements; glycoproteins; fibronectin; peptides and proteins; carbohydrates (both simple and/or complex); proteoglycans; antiangiogenins; antigens; lipids or liposomes; oligonucleotides (sense and/or antisense DNA and/or RNA); and gene therapy reagents. In other embodiments of the present invention, the backing layer and/or the internal support layer, if present, may contain one or more supplements. According to certain preferred embodiments of the present invention, the therapeutic supplement is present in an amount greater than its solubility limit in fibrin.

The following examples are illustrative only and are not intended to limit the scope of the invention as defined by the appended claims. It will be apparent to those skilled in the art that various modifications and variations can be made in the methods of the present invention without departing from the spirit and scope of the invention. Thus, it is intended that the present invention cover the modifications and variations of this invention provided they come within the scope of the appended claims and their equivalents.

### EXAMPLES

The ability of the dressings to seal an injured blood vessel was determined by an ex vivo porcine arteriotomy (EVPA) performance test, which was first described in U.S. Pat. No. 6,762,336. The EVPA performance test evaluates the ability of a dressing to stop fluid flow through a hole in a porcine artery. While the procedure described in U.S. Pat. No. 6,762,336 has been shown to be useful for evaluating haemostatic dressings, it failed to replicate faithfully the requirements for success in vivo. More specifically, the procedure disclosed in U.S. Pat. No. 6,762,336 required testing at 37° C., whereas, in the real world, wounds are typically cooler than that. This decreased temperature can significantly reduce the rate of fibrin formation and its haemostatic efficacy in trauma victims. See, e.g., Acheson et al., J. Trauma 59:865-874 (2005). The test in U.S. Pat. No. 6,762,336 also failed to require a high degree of adherence of the dressing to the injured tissue. A failure mode in which fibrin forms but the dressing fails to attach tightly to the tissue would, therefore, not be detected by this test. Additionally, the pressure utilized in the procedure (200 mHg) may be exceeded during therapy for some trauma patients. The overall result of this is that numerous animal tests, typically involving small animals (such as rats and rabbits), must be conducted to accurately predict dressing performance in large animal, realistic trauma studies and in the clinical environment.

In order to minimize the amount of time and the number of animal studies required to develop the present invention, an improved ex vivo testing procedure was developed. To accomplish this, the basic conditions under which the dressing test was conducted were changed, and the severity of the test parameters was increased to include testing at lower temperatures (i.e. 29-33° C. vs. 37° C., representing the real physiologic challenge at realistic wound temperatures (Acheson et al., J. Trauma 59:865-874 (2005)), higher pressures (i.e. 250 mmHg vs. 200 mmHg), a longer test period (3 minutes vs. 2 minutes) and larger sized arterial injuries (U.S. Pat. No. 6,762,336 used an 18 gauge needle puncture, whereas the revised procedure used puncture holes ranging from 2.8 mm to 4 mm×6 mm).

In addition, a new test was derived to directly measure adherence of the dressing to the injured tissue. Both these tests showed greatly improved stringency and are thus capable of surpassing the previous ex vivo test and replacing many in vivo tests for efficacy.

The following is a list of acronyms used in the Examples below:
CFB: Complete Fibrinogen Buffer (100 mM Sodium Chloride, 1.1 mM Calcium Chloride, 10 mM Tris, 10 mM Sodium Citrate, 1.5% Sucrose, Human Serum Albumin (80 mg/g of total protein) and Tween™ 80 (animal source) 15 mg/g total protein)
CTB: Complete Thrombin Buffer (150 mM Sodium Chloride, 40 mM Calcium Chloride, 10 mM Tris and 100 mM L-Lysine with the addition of HSA at 100 ug/ml)
ERL: Enzyme Research Laboratories
EVPA: Ex Vivo Porcine Arteriotomy
FD: Inventive haemostatic dressing
HSA: Human Serum Albumin
HD: A “sandwich” fibrin sealant haemostatic dressing as disclosed in U.S. Pat. No. 6,762,336
IFB: Incomplete Fibrinogen Buffer; CFB without HSA and Tween
PETG: Glycol-modified Polyethlylenetetrapthalate
PPG: Polypropylene
PVC: Poly vinyl chloride
TRIS: trishydroxymethylaminomethane (2-amino-2-hydroxymethyl-1,3-propanediol)

### Example 1

Backing material (DEXON™) was cut and placed into each PETG 2.4×2.4 cm mold. Twenty-five microliters of 2% sucrose was pipetted on top of each of the four corners of the backing material. Once completed the molds were placed in a −80° C. freezer for at least 60 minutes. Fibrinogen (Enzyme Research Laboratories™) was formulated in CFB. The final pH of the fibrinogen was 7.4±0.1. The fibrinogen concentrations were adjusted to 37.5, 31.7, 25.9, 20.16, 14.4, 8.64, and 4.3 mg/ml. When 2 ml of fibrinogen was delivered into the molds, this would result in a fibrinogen dose of 13, 11, 9, 7, 5, 3 or 1.5 mg/cm². Once prepared the fibrinogen was placed on ice until use. Thrombin was formulated in CTB. The final pH of the thrombin was 7.4±0.1. The concentrations of thrombin were adjusted so that when mixed with the fibrinogen solutions as described below, the combination would produce a solution that contained 0.1 units/mg of Fibrinogen. Once prepared the thrombin was placed on ice until use. The temperature of the fibrinogen and thrombin prior to dispensing was 4° C.±2° C. Molds were removed from the −80° C. freezer and placed on a copper plate that was placed on top of dry ice. A repeat pipettor was filled with fibrinogen and second repeat pipettor was filled with thrombin. Two ml of fibrinogen and 300 micro liters of thrombin were dispensed simultaneously into each mold. Once the molds were filled they were allowed to freeze and then returned to the −80° C. freezer for at least two hours. The frozen dressings were then placed into a pre-cooled Genesis™ lyophylizer (Virtis, Gardiner, N.Y.). The chamber was sealed and the temperature equilibrated. The chamber was then evacuated and the dressings lyophilized via a primary and secondary drying cycle.

The dressings were removed from the lyophylizer, sealed in foil pouches and stored at room temperature until testing. Subsequently, the dressings were evaluated in the EVPA, Adherence and Weight Assays.

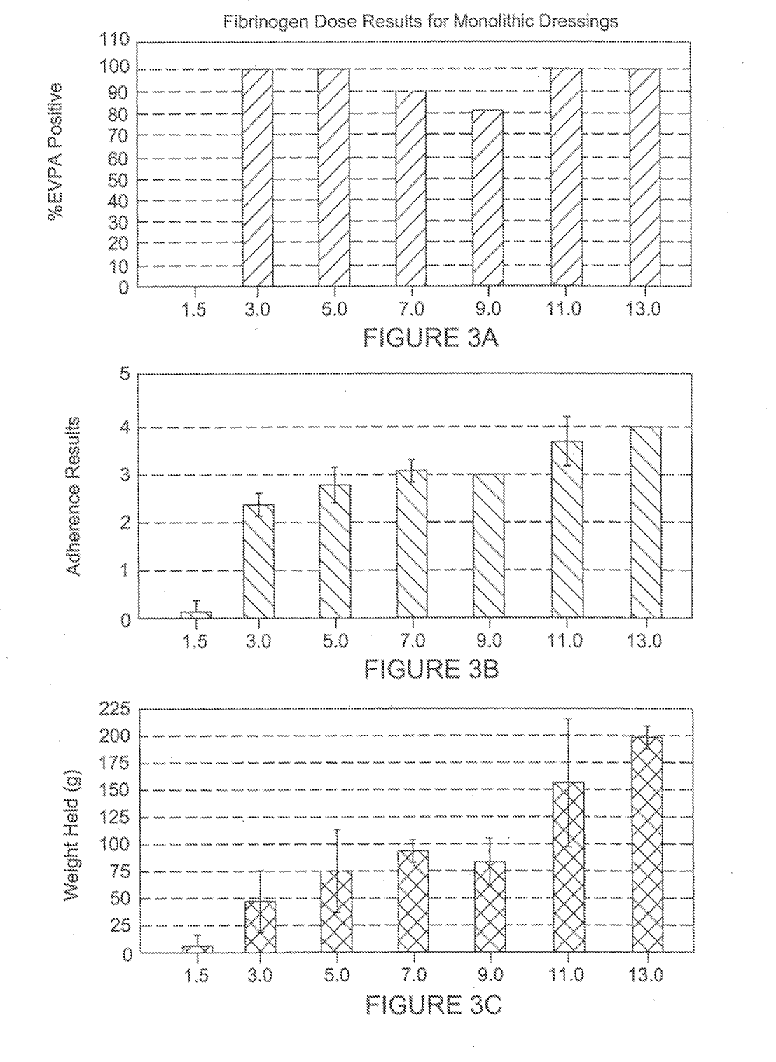

The results are given in the following Table and depicted graphically in FIGS. 3A-3C.

| | | | | Weight | Weight |
|---|---|---|---|---|---|
| | EVPA | Peel Test | Adherence | Held | Held |
| Group | Pass/Total | Adherence | Std Dev | (mean) (g) | Std Dev |
| 13 mg/cm² | 6/6 | 4.0 | 0.0 | 198.0 | 12.6 |
| 11 mg/cm² | 6/6 | 3.8 | 0.4 | 163 | 48.5 |
| 9 mg/cm² | 5/6 | 3.0 | 0.0 | 88 | 20.0 |
| 7 mg/cm² | 6/6 | 3.2 | 0.4 | 93 | 17.6 |
| 7 mg/cm² | 5/6 | 3.0 | 0.0 | 94.7 | 8.2 |
| 5 mg/cm² | 5/5 | 2.8 | 0.4 | 76 | 34.2 |
| 3 mg/cm² | 5/5 | 2.4 | 0.5 | 48 | 27.4 |
| 1.5 mg/cm² | 0/6 | 0.1 | 0.2 | 4.7 | 11.4 |

### Example 2

Monolithic dressings were manufactured as follows: backing material was cut and placed into each PETG 2.4×2.4 cm mold. Twenty-five microliters of 2% sucrose was pipetted on top of each of the four corners of the backing material. Once completed the molds were placed in a −80° C. freezer for at least 60 minutes.

For all dressings, ERL fibrinogen lot 3114 was formulated in CFB. The final pH of the fibrinogen was 7.4±0.1. The fibrinogen concentration was adjusted to 37.5 mg/ml. Once prepared the fibrinogen was placed on ice until use. Thrombin was formulated in CTB. The final pH of the thrombin was 7.4±0.1. The thrombin was adjusted to deliver 0.1 units/mg of Fibrinogen or 25 Units/ml thrombin. Once prepared the thrombin was placed on ice until use. The temperature of the fibrinogen and thrombin prior to dispensing was 4° C.±2° C. Molds were removed from the −80° C. freezer and placed on a copper plate that was placed on top of dry ice. A repeat pipettor was filled with fibrinogen and second repeat pipettor was filled with thrombin. Simultaneously 2 ml of fibrinogen and 300 micro liters of thrombin were dispensed into each mold. Once the molds were filled they were returned to the −80° C. freezer for at least two hours before being placed into the freeze dryer. Dressings were then lyophilized as described above. Once complete the dressings were stored in low moisture transmission foil bags containing 5 grams of desiccant.

Trilayer dressings were manufactured as described previously′, using the same materials as described above. Subsequently, the dressings were placed under conditions of 100% relative humidity at 37° C. for various times in order to increase their relative moisture content to desired levels. The dressings were evaluated visually and for their handling and other physical characteristics. Following this evaluation, a sample of each of the dressings was tested to determine their moisture content The remaining dressings were performance tested in the EVPA, Adherence and Weight Held assays.

### Results

The results of the assays are given in the Tables below:

TABLE 1
Performance Data of Inventive Solid Dressings

| Exposure Time | | | | Weight |
|---|---|---|---|---|
| to 100% Humidity | | | Peel Test | Held (g) |
| @37° C. | % | EVPA # | Adherence | (mean ± Std. |
| (minutes) | Moisture | Pass/Total | (±Std. Dev.) | Dev.) |
| 0 | 2.5 | 2/2 | 4.0 ± 0 | 148 ± 28.3 |
| 1 | 5.8 | 2/2 | 3.5 ± 0.71 | 123 ± 7.1 |
| 15 | 16 | 2/2 | 2.5 ± .71 | 108 ± 14.1 |
| 45 | 24 | 2/2 | 4.0 ± 0 | 168 ± 0 |
| 60 | 28 | 2/2 | 4.0 ± 0 | 273 ± 7.1 |
| 225 | 44 | 2/2 | 2 ± 0 | 58 ± 14.1 |
| 1200 | 52 | ND | ND | ND |

TABLE 2
Performance Data for Tri-layer Dressings

| Exposure Time to | | | | |
|---|---|---|---|---|
| 100% Humidity | | | | |
| @37° C. | % | EVPA # | Peel Test | Weight Held (g) |
| (minutes) | Moisture | Pass/Total | Adherence | (mean) |
| 0 | 3 | 1/1 | 2.0 | 78 |
| 15 | 22 | 1/1 | 2.0 | 78 |
| 60 | 33.7 | 0/1 | 0.5 | 28 |

TABLE 3
Integrity and Handling Characteristics of Inventive Solid Dressings

| Exposure | | | | | | | |
|---|---|---|---|---|---|---|---|
| Time | | | | | During Hydration | | |
| to 100% | | | | | | Force | |
| Humidity | Prior To Hydration | | | | | Required | After |
| @37° C. | Surface | | | | Speed of | for | Hydration |
| (minutes) | Appearance | Curling | Integrity | Flexible | Hydration | Hydration | Appearance |
| 0 | Normal | No | Excellent | No | Normal | No | Normal |
| | (Smooth, | | (No | | | | |
| | No “skin”) | | cracks or | | | | |
| | | | flaking | | | | |
| | | | off) | | | | |
| 1 | Normal | ″ | Excellent | Yes | ″ | ″ | ″ |
| | (Smooth, | | (No | | | | |
| | No “skin”) | | cracks or | | | | |
| | | | flaking | | | | |
| | | | off) | | | | |
| 15 | Normal | ″ | Excellent | ″ | ″ | ″ | ″ |
| | (Smooth, | | (No | | | | |
| | No “skin”) | | cracks or | | | | |
| | | | flaking | | | | |
| | | | off) | | | | |
| 45 | Normal | ″ | Excellent | ″ | ″ | ″ | ″ |
| | (Smooth, | | (No | | | | |
| | No “skin”) | | cracks or | | | | |
| | | | flaking | | | | |
| | | | off) | | | | |
| 60 | Normal | Slight | Excellent | ″ | ″ | ″ | ″ |
| | (Smooth, | | (No | | | | |
| | No “skin”) | | cracks or | | | | |
| | | | flaking | | | | |
| | | | off) | | | | |
| 225 | Normal | Yes | Excellent | ″ | ″ | ″ | ″ |
| | (Smooth, | | (No | | | | |
| | No “skin”) | | cracks or | | | | |
| | | | flaking | | | | |
| | | | off) | | | | |
| 1200 | Normal | Curling | Excellent | ″ | n/d | n/d | Mottled and |
| | (Smooth, | up on | (No | | | | lumpy |
| | No “skin”) | itself | cracks or | | | | |
| | | | flaking | | | | |
| | | | off) | | | | |

TABLE 4
Integrity and Handling Characteristics of Trilayer Dressings

| Exposure | | | | | | | |
|---|---|---|---|---|---|---|---|
| Time | | | | | During Hydration | | |
| to 100% | | | | | | Force | |
| Humidity | Prior To Hydration | | | | | Required | After |
| @37° C. | Surface | | | | Speed of | for | Hydration |
| (minutes) | Appearance | Curling | Integrity | Flexible | Hydration | Hydration | Appearance |
| 0 | Normal | No | Good; | No | Normal | No | Normal |
| | | | some | | | | |
| | | | delamination | | | | |
| 15 | Irregular | No | Good; | Yes | Slow | No | Mottled |
| | | | some | | | | |
| | | | delamination | | | | |
| 60 | Skinned | Yes | Good; | Yes | Very | Yes | Very |
| | | | some | | Slow | | Mottled |
| | | | delamination | | | | and lumpy |

### Conclusions:

The monolithic dressings were fully functional at very high levels of moisture. As much as 28% moisture was found to retain complete functionality. When the moisture levels rose to 44%, the dressings were still functional, however some of their activity was reduced Higher levels of moisture may also retain some function. The original dressings, at 2.5% moisture content, were not flexible, but had all the other desired properties including appearance, a flat surface, integrity, rapid and uncomplicated hydration and a smooth appearance post hydration. Once the moisture content was increased to 5.8%, the monolithic dressings became flexible, while retaining their functionality and desirable characteristics. They retained their flexibility, without curling or losing their integrity or appearing to form excessive amounts of fibrin prior to hydration.

This contrasted with the tri-layer dressings, which began to lose their desirable characteristics upon the addition of moisture, and lost them entirely by the time moisture had increased to 33%. At no time did these dressings become flexible.

### Example 3

For dressings utilizing a backing, the backing material was cut and placed into each PETG 2.4×2.4 cm mold. Twenty-five microliters of 2% sucrose was pipetted on top of each of the four corners of the backing material. Once completed the molds were placed in a −80° C. freezer for at least 60 minutes. For dressings without backing material, PETG 2.4×2.4 cm molds were placed in a −80° C. freezer for at least 60 minutes.

For all dressings, ERL fibrinogen lot 3114 was formulated in CFB. The final pH of the fibrinogen was 7.4±0.1. The fibrinogen concentration was adjusted to 37.5 mg/ml. Once prepared the fibrinogen was placed on ice until use. Thrombin was formulated in CTB. The final pH of the thrombin was 7.4±0.1. The thrombin was adjusted to deliver 0.1 units/mg of Fibrinogen or 25 Units/ml thrombin. Once prepared the thrombin was placed on ice until use. The temperature of the fibrinogen and thrombin prior to dispensing was 4° C.±2° C. Molds were removed from the −80° C. freezer and placed on a copper plate that was placed on top of dry ice. A repeat pipettor was filled with fibrinogen and second repeat pipettor was filled with thrombin. Simultaneously 2 ml of fibrinogen and 300 micro liters of thrombin were dispensed into each mold. Once the molds were filled they were returned to the −80° C. freezer for at least two hours before being placed into the freeze dryer. Dressings were then lyophylized as described below.

Both groups were performance tested in the EVPA assay. In addition, the group which had a backing was also tested in the Adherence and Weight Held assays.

### Results:

| | | | | | Weight |
|---|---|---|---|---|---|
| | EVPA # | Peel Test | Adherence | Weight Held | Held |
| Group | Pass/Total | Adherence | Std Dev | (mean) (g) | Std Dev |
| Backing | 6/6 | 3.7 | 0.5 | 153 | 37.3 |
| No Backing | 9/12 | | | | |

### Conclusions:

Dressings formulated with backing material performed well, with all dressings passing the EVPA test, and high values for adherence and weight held. Dressings without backing material were not quite as effective in the EVPA assay, however, surprisingly 75% of them passed the EVPA test. Without the backing the other tests could not be performed. The ability of the dressings made without a backing to succeed in the EVPA assay indicates that these dressings would be effective in treating arterial injuries and even more effective in treating venous and small vessel injuries.

### Example 4

For all dressings, ERL fibrinogen lot 3130 was formulated in CFB. The final pH of the fibrinogen was 7.4±0.1. The fibrinogen concentration was adjusted to 37.5 mg/ml. Once prepared the fibrinogen was placed on ice until use. Thrombin was formulated in CTB. The final pH of the thrombin was 7.4±0.1. The thrombin was adjusted to deliver 0.1 units/mg of Fibrinogen or 25 Units/ml thrombin. For the group with shredded VICRYL™ mesh dispersed within, this support material was cut into approximately 1 mm×1 mm pieces and dispersed within the thrombin solution prior to filling the molds. Once prepared the thrombin was placed on ice until use. The temperature of the fibrinogen and thrombin prior to dispensing was 4° C.±2° C. Cylindrical molds made of 10 or 3 mL polypropylene syringes (Becton Dickinson) with the luer-lock end removed were used. The plungers were withdrawn to the 6 mL and 2 mL mark respectively. For dressings utilizing a backing, the support material was cut and placed into each mold and pushed down until it was adjacent to the plunger. Once prepared the molds were placed upright and surrounded by dry ice, leaving the opening exposed at the top. 1 ml of fibrinogen and 0.15 mL of thrombin (with or without backing material dispersed within) were dispensed into the 10 mL molds and 1 ml of fibrinogen and 0.15 mL of thrombin (with or without support material dispersed within) were dispensed into the 3 mL molds, which were allowed to freeze for 5 minutes. The molds were then placed into the −80° C. freezer for at least two hours before being placed into the freeze dryer and lyophylized as described above.

Upon removal from the lyophylizer, both groups were performance tested in a modified EVPA assay. Briefly, a plastic foam form was slipped over the artery. This covering had a hole in it that corresponded to the hole in the artery and the surrounding tissue. Warm saline was added to the surface of the dressing and the mold was immediately passed down thru the hole in the foam to the artery surface. The plunger was then depressed and held by hand for 3 minutes, after which the mold was withdrawn as the plunger was depressed further. At this point the artery was pressurized and the assay continued as before.

### Results

| | | EVPA Result | Maximum |
|---|---|---|---|
| Support Material | Mold Size | (@250 mmHg) | Pressure |
| None | 10 ml | Pass | >250 mmHg |
| Dexon Mesh Backing | 10 ml | Pass | ″ |
| ″ | 3 ml | Pass | ″ |
| Shredded Dexon | 10 ml | Pass | ″ |
| Mesh (Dispersed) | | | |
| Shredded Dexon | 3 ml | Fail | 150 mmHg |
| Mesh (Dispersed) | | | |

### Conclusions:

Dressings that included no backing or a DEXON™ mesh backing performed well, with all passing the EVPA test at 250 mmHg. When the support material was dispersed throughout the composition, the dressings also performed well, with the large size (10 mL mold) dressings holding the full 250 mmHg of pressure, while the smaller held up to 150 mmHg of pressure. This indicates that the use of a support material may be optional, and its location may be on the ‘back’ of the dressing, or dispersed thou the composition, as desired.

### Example 5

Dressings made with a support material on the “back” (i.e. the non-wound-facing side) of the dressing were manufactured by first cutting the mesh support material and placing it into each PETG 10×10 cm mold. Twenty-five microliters of 2% sucrose was pipetted on top of each of the four corners of the backing material. Once completed the molds were placed in a −80° C. freezer for at least 60 minutes.

For dressings made with a support material on the “front” (i.e. the wound-facing side) of the dressing, these were manufactured without any support material in the mold. The support mesh was placed atop the dressing immediately after dispensing of the fibrinogen and thrombin into the mold (see below), and lightly pressing it into the surface prior to its freezing. In all other ways the manufacture of the dressings was similar as described below.

For all dressings, ERL fibrinogen lot 3114 was formulated in CFB. The final pH of the fibrinogen was 7.4±0.1. The fibrinogen concentration was adjusted to 37.5 mg/ml. Once prepared the fibrinogen was placed on ice until use. Thrombin was formulated in CTB. The final pH of the thrombin was 7.4±0.1. The thrombin was adjusted to deliver 0.1 units/mg of Fibrinogen or 25 Units/ml thrombin. Once prepared the thrombin was placed on ice until use. The thrombin was adjusted to deliver 0.1 units/mg of Fibrinogen or 25 Units/ml thrombin. Once prepared the thrombin was placed on ice until use. The temperature of the fibrinogen and thrombin prior, to dispensing was 4° C.±2° C. The mold was removed from the −80° C. freezer and placed on an aluminum plate that was placed on top of dry ice. The aluminum plate had a 0.25 inch hole drilled in the center and a fitting attached so that a piece of tubing could be attached to a vacuum source. The mold was centered over the hole in the aluminum plate and vacuum was turned on. The vacuum served two purposes it prevented the mold from moving and it held it flat against the aluminum plate. Thirty-five milliliters of fibrinogen and 5.25 milliliters of Thrombin were placed in 50 ml test tube, inverted three times and poured into the mold. Once the molds were filled and the support material applied as described above, they were returned to the −80° C. freezer for at least two hours before being placed into the freeze dryer. Dressings were then lyophylized as described previously.

Both groups were performance tested in the EVPA assay. In addition, the group which had a backing was also tested in the Adherence and Weight Held assays.

### Results:

| Support | | | | | |
|---|---|---|---|---|---|
| Material | | | | Weight | Weight |
| (Mesh) | EVPA # | Adherence | Adherence | Held | Held |
| Orientation | Pass/Total | Test Score | Std Dev | (mean) (g) | Std Dev |
| Back (away | 6/6 | 3.5 | 0.5 | 136 | 49 |
| from injury | | | | | |
| site) | | | | | |
| Front | 6/6 | 3.8 | 0.4 | 163 | 32 |
| (immediately | | | | | |
| adjacent to | | | | | |
| injury site) | | | | | |

### Conclusions:

Dressings formulated with backing material in either orientation well, with all dressings passing the EVPA test, and high values for adherence and weight held. This indicates that the location of a support material may be on the ‘back’ of the dressing, or the ‘front’, of the composition as desired.

### Example 6

Backing material (DEXON™) was placed into 2.4×2.4 cm PETG molds. Twenty-five microliters of 2% sucrose was pipetted on top of each of the four corners of the backing material. Once completed the mods were placed in a −80° C. freezer for at least 60 minutes.

Fibrinogen (Enzyme Research Laboratories™ (ERL) lot 3114) was formulated in CFB. The fibrinogen concentration was adjusted to 37.5 mg/ml using CFB. The final pH of the fibrinogen was 7.4±0.1. Once prepared the fibrinogen was placed on ice until use.

Thrombin was formulated in CTB. The final pH of the thrombin was 7.4±0.1. The thrombin concentrations were adjusted with CFB to produce 12.5 units/mg of Fibrinogen (upon mixing), which corresponded to 3120 Units/ml thrombin prior to mixing. Once prepared the thrombin was placed on ice until use.

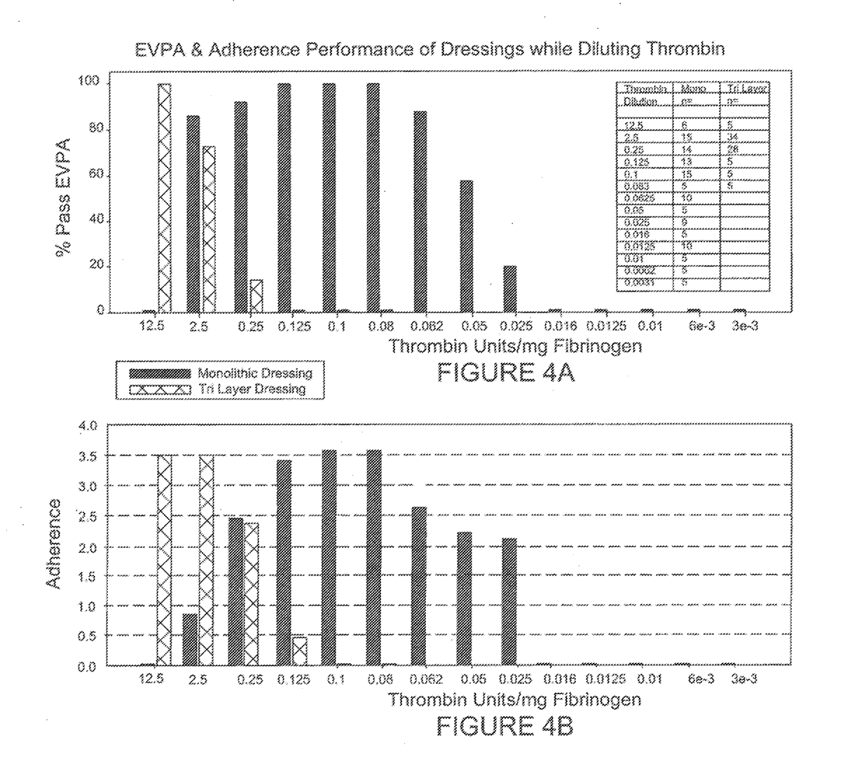

The temperature of the fibrinogen and thrombin prior to dispensing was 4° C.±2° C. Molds were removed from the −80° C. freezer and placed on a copper plate that was precooled on top of dry ice. A repeat pipettor was filled with fibrinogen and second repeat pipettor was filled with thrombin. Two ml of fibrinogen and 300 micro liters of thrombin were dispensed simultaneously into each mold. Once the molds were filled they were returned to the −80° C. freezer for at least two hours before being placed into the freeze dryer. They were then lyophilized as described below, and performance tested using the EVPA and Adherence Assays as described below. The results are shown in FIGS. 4A and 4B.

### Example 7

Backing material was placed into each 1.5×1.5 cm PVC molds. Fifteen microliters of 2% sucrose was pipetted on top of each of the four corners of the backing material. A second piece of PETG plastic was fitted on top of the 1.5×1.5 molds and held in place. This formed a closed mold. The molds were then placed in a −80° C. freezer for at least 60 minutes. Fibrinogen (ERL lot 3100) was formulated in CFB. The fibrinogen concentration was adjusted to 37.5 mg/ml using CFB. The final pH of the fibrinogen was 7.4±0.1. Once prepared the fibrinogen was placed on ice until use. Thrombin was formulated in CTB. The final pH of the thrombin was 7.4±0.1. The thrombin concentrations were adjusted using CTB to deliver the following amounts 2.5, 0.25, 0.1, 0.05, 0.025, 0.016, 0.0125 and 0.01 units/mg of Fibrinogen (upon mixing), which corresponded to 624, 62.4, 25, 12.5, 6.24, 3.99, 3.12, and 2.5 Units/ml thrombin prior to mixing. Once prepared the thrombin was placed on ice until use. The temperature of the fibrinogen and thrombin prior to dispensing was 4° C.±2° C. Molds were then removed from the −80° C. freezer and placed on an aluminum plate that was pre-cooled on top of dry ice. Three holes were punched at the top of the mold using an 18 gauge needle. One hole was used for injecting fibrinogen, the second for injecting thrombin, and the third hole served as a vent to release air that was displaced from inside the mold. A pipette was then filled with fibrinogen and a second pipette with thrombin. Simultaneously 0.78 ml of fibrinogen and 0.17 ml of thrombin were injected via these pipettes into each mold. Once filled the molds were placed on top of a pool of liquid nitrogen for thirty seconds and then returned to the −80° C. freezer for at least two hours before being placed into the freeze dryer. They were then lyophilized as described below, and performance tested using the EVPA and Adherence Assays as described below.

### Example 8

Backing material was placed into 2.4×2.4 cm PVC molds. Twenty-five microliters of 2% sucrose was pipetted on top of each of the four corners of the backing material. Once completed the molds were placed in a −80° C. freezer for at least 60 minutes. Fibrinogen (ERL lot 3100) was formulated in CFB. The fibrinogen concentration was adjusted to 37.5 mg/ml using CFB. The final pH of the thrombin was 7.4±0.1. Once prepared the fibrinogen was placed on ice until use. Thrombin was formulated in CTB. The final pH of the thrombin was 7.4±0.1. Using CTB, the thrombin concentrations were adjusted to deliver the following amounts 0.125, 0.025, 0.0125, 0.00625 and 0.0031 units/mg of Fibrinogen upon mixing, which corresponded to 31.2, 6.24, 3.12, 1.56 and 0.78 Units/ml thrombin prior to mixing. Once prepared the thrombin was placed on ice until use. The temperature of the fibrinogen and thrombin prior to dispensing was 4° C.±2° C. The molds were removed from the −80° C. freezer and placed on an aluminum plate that that was precooled on top of dry ice. A 3 ml syringe fitted with an 18 gauge needle was filled with 2 ml of fibrinogen and a second, 1 ml, syringe fitted with a 22 gauge needle was filled with 0.3 ml of thrombin. The contents of both syringes were dispensed simultaneously into each mold. Once filled the molds were placed on top of liquid nitrogen for thirty seconds and then returned to the −80° C. freezer for at least two hours before being placed into the freezer dryer. They were then lyophilized as described below, and performance tested using the EVPA and Adherence Assays as described below.

### Example 9

Backing material was placed into PVC 2.4×2.4 cm molds. Twenty-five microliters of 2% sucrose was pipetted on top of each of the four corners of the backing material. Once completed the molds were placed in a −80° C. freezer for at least 60 minutes. A vial containing 3 grams of Fibrinogen (Sigma™ Lot#3879) was removed the −20° C. freezer and placed at 4° C. for 18 hours. The bottle was then removed from the freezer and allowed to come to room temperature for 60 minutes. To the bottle, 60 ml of 37° C. water was added and allowed to mix for 15 minutes at 37° C. Once in solution the fibrinogen was dialyzed against incomplete fibrinogen buffer (IFB, which was CFB without HSA and Tween™) for 4 hours at room temperature. At the end of the four hours HSA was added to a concentration of 80 mg/g of total protein, and Tween™ 80 (animal source) was added to a concentration of 15 mg/g total protein. The final pH of the fibrinogen was 7.4±0.1. The fibrinogen concentration was then adjusted to 37.5 mg/m with CFB. Once prepared the fibrinogen was placed on ice until use. Thrombin was formulated in CTB. The final pH of the thrombin was 7.4±0.1. Using CTB, the thrombin concentrations were adjusted to deliver the following amounts 2.5, 0.25, 0.125, 0.083 and 0.0625 units/mg of Fibrinogen (upon mixing) which corresponded to 624, 62.4, 31.2, 20.8 and 15.6 Units/ml thrombin prior to mixing. Once prepared the thrombin was placed on ice until use. The temperature of the fibrinogen and thrombin prior to dispensing was 4° C.±2° C. Molds were removed from the −80° C. freezer and placed on an aluminum plate that was that was precooled on top of dry ice. A 3 ml syringe fitted with an 18 gauge needle was filled with 2 ml of fibrinogen and a second 1 ml syringe fitted with a 22 gauge needle was filled with 0.3 ml of thrombin. The contents of both syringes were dispensed simultaneously into each mold. Once filled the molds were placed on top of liquid nitrogen for thirty seconds and then returned to the −80° C. freezer for at least two hours before being placed into the freeze dryer. They were then lyophilized as described below, and performance tested using the EVPA and Adherence Assays as described below.

### Example 10

Backing material was placed in 2.4×2.4 cm PVC molds. Twenty five microliters of 2% sucrose was pipetted on top of each of the four corners of the backing material. A second piece of PETG plastic was cut to fit on top of the molds and held in place by clips located at each end of the mold, producing closed molds. Once completed the molds were placed in a −80° C. freezer for at least 60 minutes. Fibrinogen (ERL lot 3060 was formulated in CFB. The final pH of the fibrinogen was 7.4±0.1. The fibrinogen concentration was adjusted to 37.5 mg/ml using CFB. Once prepared the fibrinogen was placed on ice until use. Thrombin was formulated in CTB. The final pH of the thrombin was 7.4±0.1. Using CTB, thrombin concentrations were adjusted to deliver the following amounts 2.5, 0.25, 0.083 and 0.062 units/mg of Fibrinogen (after mixing), which corresponded to 624, 62.4, 31.2, 20.8, and 15.6 Units/ml thrombin (prior to mixing). Once prepared the thrombin was placed on ice until use. The temperature of the fibrinogen and thrombin prior to dispensing was 4° C.±2° C. Molds were removed from the −80° C. freezer and placed on an aluminum plate that was that was precooled on top of dry ice. A 3 ml syringe fitted with an 18 gauge needle was filled with 2 ml of fibrinogen and a second, 1 ml, syringe fitted with a 22 gauge needle was filled with 0.3 ml of thrombin. The contents of both syringes were dispensed simultaneously into each mold. Once filled the molds were placed on top of liquid nitrogen for thirty seconds and then returned to the −80° C. freezer for at least two hours before being placed into the freeze dryer. They were then lyophilized as described below, and performance tested using the EVPA and Adherence Assays as described below.

### Example 11

Backing material was placed into 2.4×2.4 cm PVC molds. Twenty-five microliters of 2% sucrose was pipetted on top of each of the four corners of the backing material. A second piece of PETG plastic was cut to fit on top of the 2.4×2.4 molds and held in place by the use of clips located at each end of the mod to create closed molds. The molds were then placed in a −80° C. freezer for at least 60 minutes. A vial containing 3 grams of Fibrinogen (Sigma Lot# F-3879) was removed the −20° C. freezer and placed at 4° C. for 18 hours. The bottle was then removed from the freezer and allowed to come to room temperature for 60 minutes. To the bottle, 60 ml of 37° C. water was added and allowed to mix for 15 minutes at 37° C. Once in solution the fibrinogen was dialyzed against IFB. At the end of the four hours HSA was added to a concentration of 80 mg/g of total protein, and Tween™ 80 (animal source) was added to a concentration of 15 mg/g total protein. The final pH of the fibrinogen was 7.4±0.1. The fibrinogen concentration was adjusted to 37.5 mg/ml using CFB. Once prepared the fibrinogen was placed on ice until use. Thrombin was formulated in CTB. The final pH of the thrombin was 7.4±0.1. Thrombin concentration was adjusted to deliver the following amounts 2.5, 0.25, 0.125, 0.1 and 0.083 units/mg of Fibrinogen (upon mixing), which corresponded to 624, 62.4, 31.2, 24.96 and 20.79 Units/ml thrombin (before mixing). Once prepared the thrombin was placed on ice until use. The temperature of the fibrinogen and thrombin prior to dispensing was 4° C.±2° C. Molds were removed from the −80° C. freezer and placed on an aluminum plate that was pre-cooled on top of dry ice. A 3 ml syringe fitted with an 18 gauge needle was filled with 2 ml of fibrinogen and a second, 1 ml, syringe fitted with a 22 gauge needle was filled with 0.3 ml of thrombin. The contents of both syringes were dispensed simultaneously into each mold. Once filled the molds were placed on top of liquid nitrogen for thirty seconds and then returned to the −80° C. freezer for at least two hours before being placed into the freeze dryer. They were then lyophilized as described below, and performance tested using EVPA and Adherence Assays as described below.

### Example 12

Backing material was placed into 2.4×2.4 cm PVC molds. Twenty-five microliters of 2% sucrose was pipetted on top of each of the four corners of the backing material. A second piece of PETG plastic was cut to fit on top of the molds and held in place by the use of clips located at each end of the mold to create closed molds. Once completed, the molds were placed in a −80° C. freezer for at least 60 minutes.

A vial containing 3 grams of Fibrinogen (Sigma™ Lot# F-3879) was removed from the −20° C. freezer and placed at 4° C. for 18 hours. The bottle was then allowed to come to room temperature for 60 minutes. To the bottle, 60 ml of 37° C. water was added and allowed to mix for 20 minutes at 37° C. Once in solution, the fibrinogen was dialyzed against IFB. At the end of the four hours, human serum albumin (HSA) was added to a concentration of 80 mg/g of total protein, and Tween™ 80 (animal source) was added to a concentration of 15 mg/g total protein. The final pH of the fibrinogen was 7.4±0.1. The fibrinogen concentration was adjusted to 37.5 mg/ml using CFB. Once prepared the fibrinogen was placed on ice until use.

Thrombin was formulated in CTB. The final pH of the thrombin was 7.4±0.1. Thrombin was adjusted to deliver the following amounts 2.5, 0.25, 0.125, 0.08 and 0.06 units/mg of Fibrinogen (after mixing), which corresponded to 624, 62.4, 31.2, 20.8 and 15.6 Units/ml thrombin (prior to mixing). Once prepared the thrombin was placed on ice until use. The temperature of the fibrinogen and thrombin prior to dispensing was 4° C.±2° C. Molds were removed from the −80° C. freezer and placed on an aluminum plate that was that was precooled on top of dry ice. A 3 ml syringe fitted with an 18 gauge needle was filled with 2 ml of fibrinogen and a second, 1 ml, syringe fitted with a 22 gauge needle was filled with 0.3 ml of thrombin. The contents of both syringes were dispensed simultaneously into each mold. Once filled the molds were placed on top of liquid nitrogen for thirty seconds and then returned to the −80° C. freezer for at least two hours before being placed into the freeze dryer. They were then lyophilized as described below, and performance tested using the EVPA and Adherence Assays as described below.

### Trilayer (Sandwich) Dressings

Trilayer dressings were produced in using the process described in U.S. Pat. No. 6,762,336, using the same sources of fibrinogen and thrombin as utilized to produce the monolithic dressings above.

### Results

The results of the EVPA and Adherence Assays are shown in FIGS. 4A and 4B, respectively.

### Conclusions (Examples 6-12):

Dressings produced with between 2.5 and 0.025 thrombin Units/mg of fibrinogen were active in both assays, while those with greater or lesser ratios of thrombin to fibrinogen were not. Significantly greater activity was seen over the range of 2.5 to 0.05 thrombin Units/mg of fibrinogen. Greatly improved performance was seen between the ranges of 0.25 to 0.062 thrombin Units/mg of fibrinogen, while optimum performance was seen between the ranges of 0.125 to 0.08 thrombin Units/mg of fibrinogen. This contrasted with the dressings produced using the process described in U.S. Pat. No. 6,762,336 which reached full performance at 12.5 thrombin Units/mg of fibrinogen, with unacceptable performance occurring as the thrombin concentration was diminished below 12.5 thrombin Units/mg of fibrinogen, with essentially no activity remaining at 1.4 thrombin Units/mg of fibrinogen. This difference in both the limits of performance and the optimum levels is all the more profound given that the performance of the trilayer dressings from U.S. Pat. No. 6,762,336 was decreased by the use of decreasing amounts of thrombin, while the dressing described herein showed an increased activity over this range.

### Example 13

Backing materials was cut and placed into each PETG 2.4×2.4 cm mold. Twenty-five microliters of 2% sucrose was pipetted on top of each of the four corners of the backing material. Once completed the molds were placed in a −80° C. freezer for at least 60 minutes. Enzyme Research Laboratories (ERL) fibrinogen lot 3114 was formulated in CFB. In addition, HSA was added to 80 mg/g of total protein and Tween 80 (animal source) was added to 15 mg/g total protein. The final pH of the fibrinogen was 7.4±0.1. The fibrinogen concentration was adjusted to 37.5 mg/ml. Once prepared the fibrinogen was placed on ice until use. Thrombin was formulated in 150 mM Sodium Chloride, 40 mM Calcium Chloride, 10 mM Tris and 100 mM L˜Lysine with the addition of Human Serum Albumin at 100 mg/ml. The final pH of the thrombin was 7.4±0.1. The thrombin was adjusted to deliver 0.1 units/mg of fibrinogen or 25 Units/ml thrombin. Once prepared the thrombin was placed on ice until use. The temperature of the fibrinogen and thrombin prior to dispensing was 4° C.±2° C. Molds were removed from the −80° C. freezer and placed on an aluminum plate that was placed on top of dry ice. A repeat pipettor was filled with fibrinogen and second repeat pipettor was filled with thrombin. Simultaneously 2 ml of fibrinogen and 300 micro liters of thrombin were dispensed into each mold. Once the molds were filled they were returned to the −80° C. freezer for at least two hours before being placed into the freeze dryer. One group of dressings was lyophilized on day 0, while the remainders were kept frozen at −80° C. A second group of dressings were lyophilized on day seven and a third group was lyophilized on day fourteen.

Once all dressings had been lyophilized, they were tested using the EVPA, Adherence, and Weight Assays described herein.

### Results:

| Days Frozen | | | | Weight | Weight |
|---|---|---|---|---|---|
| Prior to | EVPA # | Peel Test | Adherence | Held | Held |
| Freeze Drying | Pass/Total | Adherence | Std Dev | (mean) (g) | Std Dev |
| 0 | 5/6 | 3.5 | 0.5 | 168.0 | 63.2 |
| 7 | 6/6 | 3.8 | 0.4 | 164.7 | 29.4 |
| 14 | 6/6 | 3.7 | 0.5 | 139.7 | 29.7 |

### Conclusions:

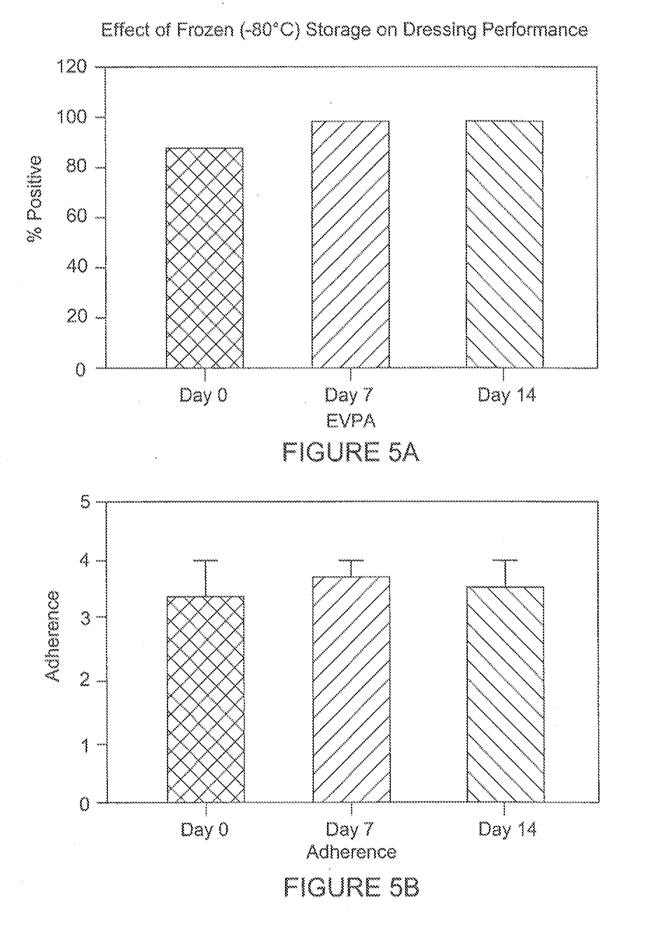

The composition of fully mixed, frozen fibrinogen and thrombin remained stable and functional for 7 and 14 days, with no apparent degradation in their performance. Longer storage would be expected to produce similar results. These results are shown graphically in FIGS. 5A and 5B.

### Example 14

Backing material was cut and placed into each PETG 2.4×2.4 cm mold. Twenty-five microliters of 2% sucrose was pipetted on top of each of the four corners of the backing material. Once completed the molds were placed in a −80° C. freezer for at least 60 minutes.

Dressing Group 1 (no Albumin, no Tween 80): Enzyme Research Laboratories (ERL) Fibrinogen lot 3130 was formulated in 100 mM Sodium Chloride, 1.1 mM Calcium Chloride, 10 mM Tris, 10 mM Sodium Citrate, and 1.5% Sucrose. The final pH of the fibrinogen was 7.4+/−0.1. The fibrinogen concentration was adjusted to 37.5 mg/ml.

Dressings Group 2 (no Albumin, Tween 80): ERL Fibrinogen was formulated in 100 mM Sodium Chloride, 1.1 mM Calcium Chloride, 10 mM Tris, 10 mM Sodium Citrate, and 1.5% Sucrose. Tween 80 (animal resource) was added 15 mg/g of total protein. The final pH of the fibrinogen was 7.4+/−0.1. The fibrinogen concentration was adjusted to 37.5 mg/ml.

Dressing Group 3 (Albumin, no Tween 80): ERL Fibrinogen was formulated in 100 mM Sodium Chloride, 1.1 mM Calcium Chloride, 10 mM Tris, 10 mM Sodium Citrate, and 1.5% Sucrose. HSA was added 80 mg/g of total protein. The final pH of the fibrinogen was 7.4+/−0.1. The fibrinogen concentration was adjusted to 37.5 mg/ml.

Dressing group 4 (Albumin, Tween 80): ERL Fibrinogen was formulated in 100 mM Sodium Chloride, 1.1 mM Calcium Chloride, 10 mM Tris, 10 mM Sodium Citrate, and 1.5% Sucrose (Fibrinogen complete buffer). In addition, HSA was added to 80 mg/g of total protein and Tween 80 (animal source) was added to 15 mg/g total protein. The final pH of the fibrinogen was 7.4+/−0.1. The fibrinogen concentration was adjusted to 37.5 mg/ml.

Once prepared, the fibrinogen solutions were placed on ice until use.

Thrombin was formulated in 150 mM Sodium Chloride, 40 mM Calcium Chloride, 10 mM Tris, 100 mM L-Lysine with the addition of HSA at 100 ug/ml. The final pH of the thrombin was 7.4+/−0.1. The thrombin was adjusted to deliver 0.1 Units/mg of fibrinogen or 25 Units/ml thrombin.

Once prepared, the thrombin solution was placed on ice until use.

The temperature of the fibrinogen and thrombin solutions prior to dispensing was 4° C.+/−2° C. Molds were removed from the −80° C. freezer and placed on an aluminum plate that was placed on top of dry ice. A repeat pipettor was filled with fibrinogen solution and second repeat pipettor was filled with thrombin solution. Simultaneously 2 ml of fibrinogen and 300 micro liters of thrombin solution were dispensed into each mold. Once the molds were filled they were returned to the −80° C. freezer for at least two hours before being placed into the freeze dryer.

### Results:

| | | | | Weight | Weight |
|---|---|---|---|---|---|
| | EVPA # | | Adherence | Held | Held |
| Formulation | Pass/Total | Adherence | Std Dev | (mean) (g) | Std Dev |
| −Alb − Tween | 0/6 | 0.8 | 1.0 | 24.0 | 26.3 |
| −Alb + Tween | 3/6 | 3.3 | 0.8 | 114.7 | 40.8 |
| +Alb − Tween | 1/6 | 1.7 | 1.0 | 45.0 | 39.9 |
| +Alb + Tween | 5/6 | 3.5 | 0.5 | 131.3 | 32.0 |

### Conclusions:

The results show that the addition of Albumin improved dressing performance. The addition of Tween improved performance even further. The combination of both resulted in the best performance.

### EVPA Performance Testing

Equipment and Supplies:
In-line high pressure transducer (Ashcroft Duralife™ or equivalent)
Peristaltic pump (Pharmacia Biotech™, Model P-1 or equivalent)
Voltmeter (Craftsman™ Professional Model 82324 or equivalent)
Computer equipped with software for recording pressure or voltage information
Tygon™ tubing (assorted sizes) with attachments
Water bath (Baxter Dutabath™ or equivalent), preset to 37° C.
Incubation chamber (VWR™, Model 1400G or equivalent), preset to 37° C.
Thermometer to monitor temperatures of both water bath and oven
Assorted forceps, hemostats, and scissors
10 cc. and 20 cc. syringes with an approximately 0.6 cm hole drilled in center and smaller hole drilled through both syringe and plunger. This hole, drilled into the end of the syringe, will be used to keep the plunger drawn back and stationary.
O-rings (size 10 and 13)
Plastic Shields to fit the 10 cc and 20 cc syringes (approximately 3.5 cm in length)
P-1000 Pipetman™ with tips
Sphygmomanometer with neonatal size cuff and bladder
Programmable Logic Controller (PLC) to control the pumps to maintain the desired pressure profile (Optional. Manual control may be used if desired.)

1. Materials and Chemicals
Porcine descending aortas (Pel-Freez Biologicals™, Catalog #59402-2 or equivalent)
Cyanoacrylate glue (Vetbond™, 3M or equivalent)
18-gauge needle(s)
0.9% Saline, maintained at 37° C.
Red food coloring
Vascular Punch(es), 2.8 mm or other
Plastic Wrap

2. Artery Cleaning and Storage
1. Store arteries at −20° C. until used.
2. Thaw arteries at 37° C. in H₂O bath.
3. Clean fat and connective tissue from exterior surface of artery.
4. Cut the arteries into ˜5 cm segments.
5. The arteries may be refrozen to −20° C. and stored until use.

3. Artery Preparation for Assay
1. Turn the artery inside-out so that the smooth, interior wall is facing outwards.
2. Stretch a size 13 O-ring over a 20 cc syringe or a size 10 O-ring over a 10 cc syringe with an approximately 0.6 cm (0.25 in) hole drilled into one side.
3. Pull the artery onto the syringe, taking care not to tear the artery or have a too loose fit. The artery should fit snugly to the syringe. Slide another O-ring of the same size onto the bottom of the syringe
4. Carefully pull both O-rings over the ends of the artery. The distance between the O-rings should be at least 3.5 cm
5. Using the blade of some surgical scissors, gently scrape the surface of the artery in order to roughen the surface of the artery.
6. Use a 18-gauge needle to poke a hole through the artery over the site of the hole in the syringe barrel (see note above)
7. The tip of the biopsy punch is inserted through the hole in the artery. Depress the punch's plunger to make an open hole in the artery. Repeat a couple of times to ensure that the hole is open and free of connective tissue.
8. Patch holes left by collateral arteries. Generally this is done by cutting a patch from a latex glove and gluing it over the hole with cyanoacrylate glue. Allow the glue to cure for at least 10 minutes.
9. Place the artery in the warmed, moistened container and place in the incubation chamber. Allow the arteries to warm for at least 30 minutes.

4. Solution and Equipment Preparation
1. Check to see that the water bath and incubation chamber are maintained at 29-33° C.
2. Make sure that there is sufficient 0.9% saline in the pump's reservoir for completion of the day's assays. Add more if needed.
3. Place 0.9% saline and 0.9% saline with a few drops of red food coloring added into containers in a water bath so that the solutions will be warmed prior to performing the assay.
4. Prepare the container for warming the arteries in the incubation chamber by lining with KimWipes™ and adding a small amount of water to keep the arteries moist.
5. Check the tubing for air bubbles, If bubbles exist, turn on the pump and allow the 0.9% saline to flow until all bubbles are removed.

5. Application of the Dressing
1. Open the haemostatic dressing pouch and remove haemostatic dressing
2. Place the haemostatic dressing, mesh backing side UP, over the hole in the artery
3. Slowly wet the haemostatic dressing with an amount of saline appropriate for the article being tested
NOTE: A standard (13-15 mg/cm² of fibrinogen) 2.4×2.4 cm haemostatic dressing should be wet with 800 μl of saline or other blood substitute. The amount of saline used can be adjusted depending on the requirements of the particular experiment being performed; however, any changes should be noted on the data collection forms.
NOTE: Wet the haemostatic dressing drop wise with 0.9% saline warmed to 29-33° C. or other blood substitute, taking care to keep the saline from running off the edges. Any obvious differences in wetting characteristics from the positive control should be noted on data collection forms.
4. Place the shield gently onto the haemostatic dressing, taking care that it lies flat between the O-rings. Press lightly to secure in place
5. Wrap the artery and haemostatic dressing with plastic wrap
6. Wrap with blood pressure cuff, taking care that the bladder is adjacent to the haemostatic dressing.
7. Pump up the bladder to 100-120 mmHg, and monitor the pressure and pump again if it falls below 100 mmHg. Maintain pressure for 5 minutes.
NOTE: Time and pressure can be altered according to the requirements of the experiment; changes from the standard conditions should be noted on the data collection forms.
8. After polymerization, carefully unwrap the artery and note the condition of the haemostatic dressing. Any variation from the positive control should be noted on the data collection form.

Exclusion Criterion:

The mesh backing must remain over the hole in the artery. If it has shifted during the polymerization and does not completely cover the hole the haemostatic dressing must be excluded.

Testing Procedure

1. Diagram of Testing Equipment Set-Up

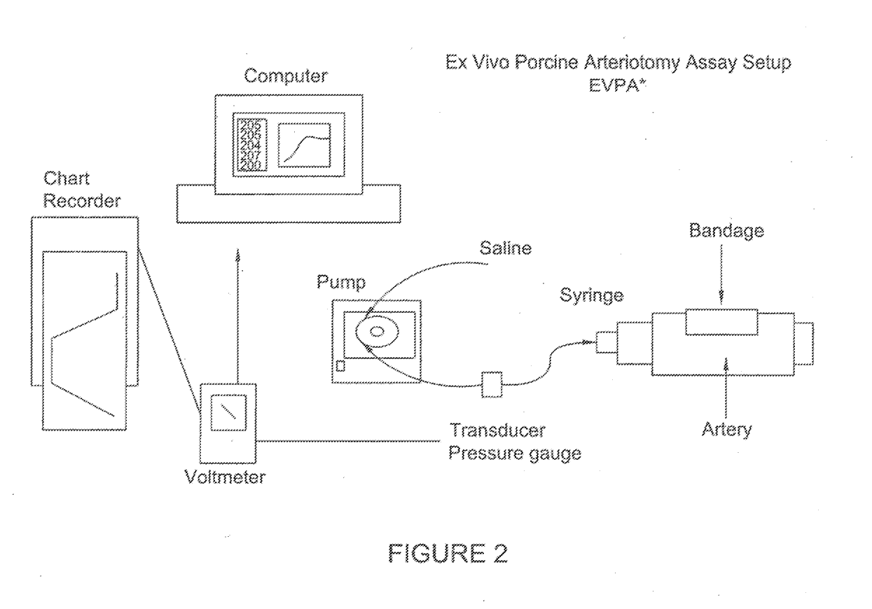

The set-up of the testing equipment is shown in FIG. 2. Some additional, unshown components may be utilized to read out (pressure gauge) or control the pressure within the system.

2. Equipment and Artery Assembly

Fill the artery and syringe with red 0.9% saline warmed to 37° C., taking care to minimize the amount of air bubbles within the syringe & artery. Filling the artery with the opening uppermost can assist with this. Attach the artery and syringe to the testing apparatus, making sure that there are as few air bubbles in the tubing as possible. The peristaltic pump should be calibrated so that it delivers approximately 3 ml/min. If available, the PLC should be operated according to a pre-determined range of pressures and hold times as appropriate for the article being tested. If under manual control, the pressure/time profile to be followed is attained by manually turning the pump on and off while referencing the system pressure as read out by one or more pressure-reading components of the system. Following the conclusion of testing, the haemostatic dressing is subjectively assessed with regard to adhesion to the artery and formation of a plug in the artery hole. Any variations from the positive control should be noted on the data collection form.

### Success Criteria

Haemostatic dressings that are able to withstand pressures for 3 minutes are considered to have passed the assay. When a haemostatic dressing has successfully passed the assay the data collection should be stopped immediately so that the natural decrease in pressure that occurs in the artery once the test is ended isn't included on the graphs. Should the operator fail to stop data collection, these points can be deleted from the data file to avoid confusing the natural pressure decay that occurs post-test with an actual dressing failure. The entire testing period from application of the haemostatic dressing to completion must fall within pre-established criteria. The maximum pressure reached should be recorded on the data collection form.
NOTE: Typical challenge is 250 mmHg for three minutes in one step, but that may be altered based on the article being tested. Changes from the standard procedure should be noted on the data collection forms.

### Failure Criteria

Haemostatic dressings that start leaking saline at any point during testing are considered to have failed the assay.
NOTE: Build failures that are caused by artery swelling can be ignored and the test continued or re-started (as long as the total testing time doesn't fall beyond the established limit).

When leakage does occur, the pressure should be allowed to fall ˜20 mmHg before data collection is stopped so that the failure is easily observed on the graphs. The pressures at which leakage occurred should be recorded on the data collection form. Should the data collection stop in the middle of the experiment due to equipment failure the data can be collected by hand at 5 second intervals until the end of the test or haemostatic dressing failure, whichever happens first. The data points should be recorded on the back of the data collection form, clearly labeled, and entered by hand into the data tables.

### Exclusion Criteria

If the total testing period exceeds the maximum allowed for that procedure, regardless of cause, results must be excluded. If there are leaks from collaterals that can't be fixed either by patching or finger pressure the results must be excluded. If the test fails because of leaks at the O-rings, the results must be excluded. If the mesh backing does not completely cover the hole in the artery, the results must be excluded.

Adherence Performance Testing

1. Equipment and Supplies

Hemostat(s), Porcine artery and haemostatic dressing (usually after completion of the EVPA Assay although it does not need to be performed to do the Adherence Assay).

2. Preparation of the Artery+Dressing

After application of the dressing without completion of the EVPA Assay, the dressing is ready for the Adherence Assay and Weight Limit Test (if applicable). After application of the dressing and subsequent EVPA Analysis, the artery and syringe system is then disconnected slowly from the pump so that solution does not spray everywhere. The warmed, red saline solution from the EVPA Assay remains in the syringe until the Adherence Assay and Weight Limit Test (if applicable) is completed.

Performance of the Adherence Assay

1. After preparation of the artery and dressing (with or without EVPA analysis), gently lift the corner of the mesh and attach a hemostat of known mass to the corner.
NOTE: If the FD developed a channel leak during the performance of the EVPA Assay, test the adherence on the opposite of the haemostatic dressing to obtain a more accurate assessment of the overall adherence.

2. Gently let go of the hemostat, taking care not to allow the hemostat to drop or twist. Turn the syringe so that the hemostat is near the top and allow the hemostat to peel back the dressing as far as the dressing will permit. This usually occurs within 10 seconds. After the hemostat has stopped peeling back the dressing, rate the adherence of the bandage according to the following scale:

| Dressing Performance Score | Amount of Adherence |
|---|---|
| 4 | 90+% |
| 3 | 75-90% |
| 2 | 50-75% |
| 1 | ~50% |
| 0.5 | Only the plug holds the hemostat |
| 0 | No adherence |

### Exclusion Criteria

The mesh backing must remain over the hole in the artery. If it has shifted during the polymerization and does not completely cover the hole the haemostatic dressing must be excluded.

### Success Criteria

Dressings that are given an adherence score of 3 are considered to have passed the assay.

### Failure Criteria

If a dressing does not adhere to the artery after application and/or prior to performing the EVPA assay, it is given a score of 0 and fails the adherence test. If a dressing receives a score <2, the dressing is considered to have failed the Adherence Assay.

### Weight Held Performance Assay

After the initial scoring of the “Adherence Test”, weights may then be added to the hemostat in an incremental manner until the mesh backing is pulled entirely off of the artery. The maximum weight that the dressing holds is then recorded as a measure of the amount of weight the dressing could hold attached to the artery.

### Moisture Assay

Moisture determinations were carried out using a Brinkman Metrohm Moisture Analyzer System. The system contains the following individual components, 774 Oven Sample Processor, 774SC Controller, 836 Titrando, 5 ml and 50 ml 800 Dosino Units and a 801 Stirrer. The system was connected to a computer using the Brinkman Tiamo software for data collection, analysis and storage. The moisture system is set-up and run according to the manufactures recommendations and specifications to measure the moisture content of lyophilized samples using the Karl Fischer method.

All components were turned on and allowed to reach operating temperature prior to use. Lactose and water were run as standards and to calibrate the instrument. Once the machine was successfully calibrated, samples were prepared as follows. Dressing pieces weighing at least 30 mg were placed into vials and capped. The vials were placed in the 774 Oven Sample Processor in numerical order, and one empty capped vial is placed in the conditioning space. The machine was then run to determine the moisture content (residual moisture) in the controls and samples.

### SDS-PAGE Gel Electrophotesis

Each dressing is cut into ¼'s, approximately 50 mg per section, and a section is then placed into a 15 mL conical tube. For the production control (ie Time 0), 1.0 mL of Okuda Dissolving Solution (10 M Urea, 0.1% Sodium Dodecyl Sulfate, 0.1% β-Mercaptoethanol) is added. For the remaining 3 pieces, 80 μL of 0.9% Saline is added to wet the dressing. The pieces are then incubated at 37° C. for 2, 5, and 10 minutes or such time as desired. To stop the reaction at the desired time, 1.0 mL of the Okuda Dissolving solution is added. The samples are then incubated at room temperature overnight, and then incubated at 70° C. for 30 minutes.

To prepare the samples for loading onto the gel, the samples which were previously dissolved in the Okuda Dissolving Solution were added to Sample buffer so that a 20 aliquot contains 10 μg. One μL of 0.1 M Dithiothreitol was then added to each sample. Twenty μL of each diluted sample is then loaded onto an 8% Tris-Glycine gel (Invitrogen), 1.0 mm thick, 10 wells. The gels were then run at 140V until the dye front reached the end of the gel. They were then removed and placed into Coomassie Blue Stain (50% v/v Methanol, 0.25% w/v Coomassie Brilliant Blue, 10% w/v Acetic Acid in ddH2O) on a shaking platform for a minimum of 1 hour. The gel is then transferred to the Destain Solution (25% Methanol, 10% Acetic Acid, 65% ddH20) on a shaking platform until the background is nearly colorless.

After destaining, the gels were scanned, and the γ-γ dimer bands and the Aα, and Bβ bands analyzed by Scion densitometry software in order to determine the amount of conversion that occurred.

In accordance with these and other objects, a first embodiment of the present invention is directed to a haemostatic composition comprising a frozen mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis. The particular amount of fibrin that may be contained in the composition may vary depending upon the ultimate intended use of the composition. Suitable “insufficient amounts” of fibrin may therefore be determined empirically by one skilled in the art, depending upon the intended use thereof.

Another embodiment of the present invention is directed to a dried haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin, upon application to or mixing with bodily fluids or an exogenous aqueous fluid, preferably containing an effective amount of Ca+2, and/or application to injured tissue, to provide effective hemostasis.

Another embodiment of the present invention is directed to a lyophilized haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

Another embodiment of the present invention is directed to a haemostatic monolithic dressing for treating wounded tissue in a patient which comprises an effective mixture of dried fibrinogen and thrombin, with or without Factor XIII, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

Another embodiment of the present invention is directed to a haemostatic monolithic dressing for treating wounded tissue in a patient which comprises an effective mixture of lyophylized fibrinogen and thrombin, with or without Factor XIII, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

Another embodiment of the present invention is directed to a haemostatic monolithic dressing for treating wounded tissue in a patient which comprises a backing material, an effective mixture of dried fibrinogen and thrombin, with or without Factor XIII, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

Another embodiment of the present invention is directed to a haemostatic monolithic dressing for treating wounded tissue in a patient which comprises a backing material, an effective mixture of lyophillized fibrinogen and thrombin, with or without Factor XIII, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

Another embodiment of the present invention is directed to a haemostatic composition for treating wounded tissue in a patient which comprises an effective mixture of fibrinogen and thrombin, with or without Factor XIII, wherein one or more components of the mixture are non-homogenously distributed throughout said mixture, and which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

Another embodiment of the present invention is directed to a haemostatic monolithic dressing for treating wounded tissue in a patient which comprises an effective mixture of fibrinogen and thrombin, with or without Factor XIII, wherein one or more components of the mixture are non-homogenously distributed throughout said mixture, and which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

Another embodiment of the present invention is directed to a haemostatic monolithic composition for treating wounded tissue in a patient which comprises an effective mixture of fibrinogen and thrombin, with or without Factor XIII, wherein one or more components of the mixture are non-homogenously distributed throughout said mixture according to a continuously varying gradient, and which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

Another embodiment of the present invention is directed to a monolithic dressing for treating wounded tissue in a patient which comprises an effective mixture of fibrinogen and thrombin, with or without Factor XIII, wherein one or more components of the mixture are non-homogenously distributed throughout said mixture according to a continuously varying gradient, and which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

Another embodiment of the present invention is directed to a haemostatic monolithic composition for treating wounded tissue in a patient which comprises an effective mixture of dried fibrinogen and thrombin, with or without Factor XIII, wherein one or more components of the mixture are non-homogenously distributed throughout said mixture, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

Another embodiment of the present invention is directed to a haemostatic monolithic dressing for treating wounded tissue in a patient which comprises an effective mixture of dried fibrinogen and thrombin, with or without Factor XIII, wherein one or more components of the mixture are non-homogenously distributed throughout said mixture, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

Another embodiment of the present invention is directed to a haemostatic composition for treating wounded tissue in a patient which comprises an effective mixture of lyophylized fibrinogen and thrombin, with or without Factor XIII, wherein one or more components of the mixture are non-homogenously distributed throughout said mixture, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

Another embodiment of the present invention is directed to a haemostatic monolithic dressing for treating wounded tissue in a patient which comprises an effective mixture of lyophylized fibrinogen and thrombin, with or without Factor XIII, wherein one or more components of the mixture are non-homogenously distributed throughout said mixture, which contains insufficient fibrin to prohibit its effective use as a haemostatic agent, and which further retains the ability to convert sufficient fibrinogen to fibrin, upon application to or mixing with bodily fluids or an exogenous aqueous fluid, and/or application to injured tissue, to provide effective hemostasis.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: filling a suitable mold with the mixture components; and applying sufficient cooling to the mold and or composition so as to freeze the mixture into a monolithic mass before the formation of sufficient fibrin to prohibit its use as a haemostatic agent occurs. One preferred method of doing this is by cooling the two active bulk substances below 0° C. and then combining the two slurries prior to dispensing into the mold and freezing. Particular ways of accomplishing this include mixing together pre-cooled fibrinogen and pre-cooled thrombin (both pre-cooled to 2°-8° C.) in a pre-cooled dispensing vessel held between 0° C. and above the freezing point of the mixed solution (the ice slurry could preferably be mixed to ensure homogeneity) and then snap frozen or the fibrinogen and thrombin could be individually pre-cooled to a temperature below 0° C. and above the freezing point of the solution to form an ice/water slurry and then the two slurries could then be mixed together (again, the ice slurries could be mixed to ensure homogeneity prior to dispensing) and then snap frozen. The parameters to be optimized include:
1. temperature and time that pre-cooled fibrinogen and thrombin are stable;
2. temperature and time Fibrinogen and thrombin ice slurries are stable;
3. temperature and time the mixed fibrinogen and thrombin ice slurries are stable; and
4. chemical additive(s) which can lower the freezing temperature of each mixture or the combined mixture.

While not wishing to be bound by any theory of operability, according to Seegers et al (Arch Biochem Biophys 128:194-201, 1968), the optimal pH for Thrombin activity is near or at pH 8.0 for turnover of chromogenic substrates, with activity found across the pH range >5 and <11 (activity reached zero at the extremes of this range). Most chromogenic assays of Thrombin activity are buffered at pH 8.3 to be at or near this optimum condition. Similar pH-dependence for clotting of fibrinogen is reported by Mihalyi et al (Biochemistry 30:4753-4762, 1991). Maximum rate was near pH 7.8, and the rate was only slightly slower at pH 8.8. While clotting assays are usually conducted at pH 7.4, this is probably done to mimic physiological conditions in the blood stream, not related to the higher pH for maximum thrombin clotting activity.

Another embodiment of the present invention is directed to a frozen haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said composition comprising the mixture of components having a pH in the range of 1-6 (i.e. >1 and <6) or having a pH >10. Particularly preferred examples of this and similar embodiments may have a pH in the range of 1-5 or a pH >11.

Another embodiment of the present invention is directed to a dried haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon exposure to an aqueous environment to provide effective hemostasis, said composition comprising the mixture of components having a pH >1 and <6 or a pH >10. As noted above, particularly preferred examples include a pH in the range of 1 to 5 or a pH >11.

Another embodiment of the present invention is directed to a lyophylized haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon exposure to an aqueous environment to provide effective hemostasis, said composition comprising the mixture of components having a pH >1 and <6 or a pH >10. As noted above, particularly preferred examples include a pH in the range of 1-5 or a pH >11.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: formulating the mixture components such that when mixed, they form a mixture having a pH >1 and <6 or a pH >10; filling a suitable mold with the mixture components; and applying sufficient cooling to the mold and/or to the mixture components so as to freeze the mixture into a monolithic mass before excess fibrin formation occurs.

Another embodiment of the present invention is directed to a method for producing a haemostatic composition comprising a dried mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: formulating the mixture components such that when mixed, they form a mixture having a pH >1 and <6 or a pH >10; filling a suitable mold with the mixture components; and applying sufficient cooling to the mold and/or to the mixture components so as to freeze the mixture into a monolithic mass before excess fibrin formation occurs, and subsequently drying the mixture.

Another embodiment of the present invention is directed to a method for producing a lyophilized haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: formulating the mixture components such that when mixed, they form a mixture having a pH >1 and <6 or a pH >10; filling a suitable mold with the mixture components; and applying sufficient cooling to the mold and/or to the mixture components so as to freeze the mixture into a monolithic mass before excess fibrin formation occurs, and subsequently lyophilizing the mixture to a suitably low residual moisture level.

In certain embodiments of the present invention, the concentration of sodium ion (Na+) may be varied. While not wishing to be bound by any theory of operability, sodium ion is known to bind to Thrombin and cause an allosteric shift from a “slow” form of Thrombin (predominating at zero or very low sodium ion concentrations) to a “fast” form at 0.2M sodium ion content. The slow form does not clot fibrinogen quickly, but activates Protein C well and thereby inhibits the coagulation process (i.e. is anticoagulant). The fast form clots fibrinogen quickly (i.e. is procoagulant), but activates Protein C poorly, and does not foster the anticoagulation system of plasma.

While not wishing to be bound by any theory of operability, in certain embodiments of the invention, the sodium content of the Thrombin and/or fibrinogen components (alone or in combination with other process variables) can be manipulated to foster or inhibit clotting. For example, low sodium content can inhibit clotting of fibrinogen as the components are mixed to form a monolithic bandage. Later, the natural concentration of components that occurs when the mixture is subjected to lyophilization can increase the sodium content to foster clotting of fibrinogen when the dressing is hydrated by application to wounded tissue.

In early work by Di Cera and colleagues, the authors used 0.2M NaCl to put thrombin into the fast form, whereas they used 0.2M choline chloride (no Na+) to study the slow form. An example can be seen in Dang Q D, Vindigni A and Di Cera E, An allosteric switch controls the procoagulant and anticoagulant activities of thrombin, Proc Natl Acad Sci USA, 92:5977-5981, 1995.

A region of Na+ concentration where this allosteric shift takes place is at the Na+ concentration of normal plasma. (For a discussion see Di Cera E, Thrombin Interactions, Chest 124 Supplement: 11S-17S, 2003). At the Na+ content in normal blood (140 mEq/L), the slow and fast forms are present at a 2:3 ratio (40% slow, 60% fast). Hypernatremia (Na+>145 mEq/L) is often associated with increased fibrinogen cleavage and venous thrombosis. Hyponatremia (Na+<135 mEq/L) has reportedly been associated with increased bleeding in infants (Di Cera2, pp14S-15S).

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: formulating the mixture components such that when mixed, they form a mixture with a low sodium content; filling a suitable mold with the mixture components; and applying sufficient, cooling to the mold and/or to the mixture components so as to freeze the mixture into a monolithic mass before excess fibrin formation occurs.

Another embodiment of the present invention is directed to a haemostatic composition comprising; a frozen mixture of fibrinogen and thrombin, with or without Factor XIII, with a low sodium content which contains insufficient fibrin to prohibit its effective use as a haemostatic agent, and which further retains the ability to convert sufficient fibrinogen to fibrin upon thawing to provide effective hemostasis, said composition comprising a mixture with a low sodium content.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: formulating the mixture components such that when mixed, they form a mixture comprising substantially no Ca+2 or Mg+2; filling a suitable mold with the mixture components; and applying sufficient cooling to the mold and/or to the mixture components so as to freeze the mixture into a monolithic mass before excess fibrin formation occurs.

Another embodiment of the present invention is directed to a haemostatic composition comprising a frozen mixture of fibrinogen and thrombin, with or without Factor XIII, said mixture further comprising substantially no Ca+2 or Mg+2; which contains insufficient fibrin to prohibit its effective use as a haemostatic agent, and which further retains the ability to convert sufficient fibrinogen to fibrin upon thawing to provide effective hemostasis.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components, said filling being conducted in the vertical direction; and applying sufficient cooling to the mold and/or to the mixture components so as to freeze the mixture into a monolithic mass before excess fibrin formation occurs.

Another embodiment of the present invention is directed to a method for producing a haemostatic composition comprising a mixture of fibrinogen and thrombin, with or without Factor XIII, which contains insufficient fibrin to prohibit its effective use as a agent, and which further retains the ability to convert sufficient fibrinogen to fibrin upon thawing to provide effective hemostasis, said method comprising the steps of: suitably formulating the mixture components; filling a suitable mold with the mixture components, said filling being conducted in the horizontal direction; and applying sufficient cooling to the mold and/or to the mixture components so as to freeze the mixture into a monolithic mass before excess fibrin formation occurs.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components; and applying sufficient convective cooling to the mold and/or to the mixture components so as to freeze the mixture into a monolithic mass before excess fibrin formation occurs.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components; and applying sufficient conductive cooling to the mold and/or to the mixture components so as to freeze the mixture into a monolithic mass before excess fibrin formation occurs.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components; and applying sufficient radiative cooling to the mold and/or to the mixture components so as to freeze the mixture into a monolithic mass before excess fibrin formation occurs.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components; and applying sufficient blast cooling to the mold and/or to the mixture components so as to freeze the mixture into a monolithic mass before excess fibrin formation occurs.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components, and applying sufficient cooling to one or more sides of the mold and/or to the mixture components so as to freeze the mixture into a monolithic mass before excess fibrin formation occurs.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components, and applying sufficient cooling to two or more opposing sides of the mold and/or to the mixture components so as to freeze the mixture into a monolithic mass before excess fibrin formation occurs.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components, and applying sufficient cooling to one or more sides of the mold and/or to the mixture components so as to freeze the mixture into a monolithic mass before excess fibrin formation occurs, said freezing occurring in the direction parallel to the shortest axis of the mold.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components, and applying sufficient cooling to one or more sides of the mold and/or to the mixture components so as to freeze the mixture into a monolithic mass before excess fibrin formation occurs, said freezing occurring in the direction parallel to the longest axis of the mold.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components, and applying sufficient cooling to one or more sides of the mold and/or to the mixture components so as to freeze the mixture into a monolithic mass before excess fibrin formation occurs, said freezing occurring in the direction parallel to the second shortest axis of the mold.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components, and applying sufficient cooling to one or more sides of the mold and/or to the mixture components so as to freeze the mixture into a monolithic mass before excess fibrin formation occurs, said freezing occurring in the directions parallel to two or more of the axes of the mold.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components, and applying sufficient cooling to one or more sides of the mold and/or to the mixture components so as to freeze the mixture into a monolithic mass before excess fibrin formation occurs, said freezing occurring in the directions parallel to all of the axes of the mold.

Another embodiment of the present invention is directed to a haemostatic monolithic dressing for treating wounded tissue in a patient which comprises an effective mixture of frozen fibrinogen and thrombin, with or without Factor XIII, which contains insufficient fibrin to prohibit its effective use as a haemostatic agent, and which further retains the ability to convert sufficient fibrinogen to fibrin, upon application to and/or mixing with a bodily fluid or an exogenous aqueous fluid and/or upon application to injured tissue, to provide effective hemostasis.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components; and applying sufficient blast cooling to the mold and/or to the mixture components so as to freeze the mixture into a monolithic mass before excess fibrin formation occurs.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; filling a suitable mold with the mixture components; and applying sufficient convective cooling with a cryogenic gas to the mold and/or to the mixture components so as to freeze the mixture into a monolithic mass before excess fibrin formation occurs.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; producing dried filaments of the components via centrifugal spinning; and combining filaments of the components into a single structure capable of producing effective hemostasis by any of the means known and available to those of skill in the art.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably co-formulating the mixture components; producing dried filaments of the co-formulated components via centrifugal spinning; and combining filaments of the components into a single structure capable of producing effective hemostasis by any of the means known and available to those of skill in the art.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; producing dried filaments of the components via electrospinning; and combining filaments of the components into a single structure capable of producing effective hemostasis by any of the means known and available to those of skill in the art.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably co-formulating the mixture components; producing dried filaments of the co-formulated components via electrospinning; and combining filaments of the components into a single structure capable of producing effective hemostasis by any of the means known and available to those of skill in the art.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; freezing said compositions, producing small fragments of the components; and combining said fragments of the components into a single structure capable of producing effective hemostasis by any of the means known and available to those of skill in the art, including, but not limited to, pressing the fragments into a single cohesive mass, with or without the addition of sufficient exogenous heat to facilitate partial melting of the fragments, followed by sufficient cooling to freeze the partially melted fragments into monolithic mass before excess fibrin formation occurs, and subsequently lyophilizing the mixture to a suitably low residual moisture level.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably co-formulating the mixture components into a single mass; freezing said mass, producing small fragments of said mass; and combining said fragments into a single structure capable of producing effective hemostasis by any of the means known and available to those of skill in the art, including, but not limited to, pressing the fragments into a single cohesive mass, with or without the addition of sufficient exogenous heat to facilitate partial melting of the fragments, followed by sufficient cooling to freeze the partially melted fragments into monolithic mass before excess fibrin formation occurs, and subsequently lyophilizing the mixture to a suitably low residual moisture level.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably formulating the mixture components; freezing said mixture components in such a manner as to simultaneously produce small fragments of the components by any of the means known to those of skill in the art, including, but not limited to, spraying the mixture in the presence of an expanding cryogenic gas, such as liquid nitrogen or compressed carbon dioxide, and combining said fragments of the components into a single structure capable of producing effective hemostasis by any of the means known and available to those of skill in the art, including, but not limited to, pressing the fragments into a single cohesive mass, with or without the addition of sufficient exogenous heat to facilitate partial melting of the fragments, followed by sufficient cooling to freeze the partially melted fragments into monolithic mass before excess fibrin formation occurs, and subsequently lyophilizing the mixture to a suitably low residual moisture level.

Another embodiment of the present invention is directed to a method for producing a haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis, said method comprising the steps of: suitably co-formulating the mixture components into a single mass; freezing said mass in a manner as to simultaneously produce small fragments of said mass by any of the means known to those of skill in the art, including, but not limited to, spraying the mixtures in the presence of an expanding cryogenic gas, such as liquid nitrogen or compressed carbon dioxide, and combining said fragments into a single structure capable of producing effective hemostasis by any of the means known and available to those of skill in the art, including, but not limited to, pressing the fragments into a single cohesive mass, with or without the addition of sufficient. exogenous heat to facilitate partial melting of the fragments, followed by sufficient cooling to freeze the partially melted fragments into monolithic mass before excess fibrin formation occurs, and subsequently lyophilizing the mixture to a suitably low residual moisture level.

Additionally, while not wishing to bound by any theory of operability, in certain embodiments of the present invention, volatile buffers can be utilized to maintain the pH of a protein solution, and can be removed from the protein when the solution is dried by lyophilization or other evaporative process. For example, a protein can be buffered at pH 5 with ammonium acetate, and upon drying the ammonium acetate evaporates and the pH reverts to that of nonvolatile buffering components (e.g., the protein itself or other constituent buffers).

Similarly, one or more of the protein components of the composition may be suspended in a volatile non-aqueous solvent, thereby partitioning it from the other components during mixing to form a monolithic composition. If this organic solvent is volatile, then it can be removed by drying, leaving all the components in a substantially organic composition that is capable or reacting to form fibrin for an effective haemostatic effect upon re-hydration.

Another embodiment of the present invention is directed to a method for producing a lyophilized haemostatic composition comprising a mixture of fibrinogen and thrombin, with or without Factor XIII, which contains insufficient fibrin to prohibit its effective use as a haemostatic agent, said composition comprising the mixture of components having a pH <6 or a pH >8 and a volatile buffer having a pH <6 (e.g. ammonium acetate) or a pH >8 (e.g. ammonium bicarbonate) which is removed by the lyophilization process, leaving a solid at neutral pH which retains the ability to convert sufficient fibrinogen to fibrin upon reconstitution to provide effective hemostasis.

The Lyotropic Series of chemicals is a ranking of chemical compounds or ions based on their effect on the structure and aggregation state of macromolecules.

At one end of the Lyotropic Series are chaotropic agents (also called “salting-in”, “structure-breaking” or “destabilizing” chemicals) that reduce the interactions between proteins or protein domains, and therefore reduce the tendency of proteins to interact or aggregate. Examples of chaotropic agents include, but are not limited to: urea, guanidine, arginine, thiocyanate, bisulfite, iodide, nitrate, calcium, magnesium, and chloride ions.

At the other end of the Lyotropic Series are “anti-chaotropes” (usually called “salting-out”, “structure making” or “stabilizing” chemicals) which tend to enhance the interaction, aggregation and/or precipitation of proteins. Anti-chaotropic anions include, but are not limited to: sulfate (e.g. ammonium sulfate), phosphate, citrate, and EDTA. Cations include quaternary amines, ammonium and to a lesser extent sodium and potassium ions.

The Lyotropic Series was first described by Von Hippel and Schleich (“The effects of neutral salts on the structure and conformational stability of macromolecules in solution”, in Structure and Stability of Biological Macromolecules, Timasheff and Fasman (eds), Vol 2, Marcel Dekker, New York, p 417-574). One clear protein application was summarized by Busby et al (J Biol Chem 256:12140-12147, 1981). U.S. Pat. No. 6,447,774 (Metzner et al) claims the use of chaotropes to stabilize liquid formulations of fibrinogen and Factor XIII, as part of a storage stable liquid fibrin sealant.

While not wishing to be bound by any theory of operability, in certain embodiments of the present invention, chaotropes may help to prevent the formation of fibrin during mixing of fibrinogen and thrombin proteins to prepare the haemostatic composition. While not wishing to be bound by any theory of operability, in certain other embodiments, anti-chaotropes may enhance these protein-protein interactions. The composition of the protein mixture can therefore be adjusted to achieve a beneficial balance between chaotropic and anti-chaotropic compounds to achieve the desired properties of the protein mixture. It is to be understood that the presence of said components must not have unacceptable deleterious affects on the fibrinogen, Factor XIII or thrombin under the selected conditions.

Another embodiment of the present invention is directed to a frozen (or dried or lyophilized) haemostatic composition comprising a mixture of fibrinogen and thrombin, with or without Factor XIII, which contains insufficient fibrin to prohibit its effective use as a haemostatic agent, and which further retains the ability to convert sufficient fibrinogen to fibrin upon thawing (or upon application to or mixing with a bodily fluid or an exogenous aqueous fluid and/or upon application to injured tissue) to provide effective hemostasis, wherein one or more of the components is a chaotropic compound.

Another embodiment of the present invention is directed to a method for producing a lyophilized haemostatic composition comprising a mixture of fibrinogen and thrombin, with or without Factor XIII, which contains insufficient fibrin to prohibit its effective use as a haemostatic agent, said composition comprising the mixture of components containing one or more chaotropic compounds together with one or more ant-chaotropic compounds, which retains the ability to convert sufficient fibrinogen to fibrin upon reconstitution to provide effective hemostasis.

Additionally, in each of the embodiments of the present invention, in addition to the active components of the mixture, the compositions and mixtures may also optionally contain one or more suitable foaming agents, such as a mixture of citric acid and sodium bicarbonate. Additional agents that generate gas when thawed and/or hydrated are known to those skilled in the art.

Each of the inventive haemostatic dressings may also further comprise a backing material on the side of the bandage opposite the wound-facing side. The backing material may be affixed with a physiologically-acceptable adhesive, or may be self-adhering (e.g., by having a sufficient surface static charge). The backing material may be a resorbable or non-resorbable material. Preferably the backing is resorbable, such as collagen, fibrin, fibrinogen, Vicryl™ or Dexon™. The backing material may be proteinacious, such as keratin, silk etc.

Any suitable resorbable material known to those skilled in the art may be employed in the present invention. For example, the resorbable material may be a proteinaceous substance, such as silk, fibrin, keratin, collagen and/or gelatin, or a carbohydrate substances, such as alginates, chitin, cellulose, proteoglycans (e.g. poly-N-acetyl glucosamine), glycolic acid polymers, lactic acid polymers, or glycolic acid/lactic acid co-polymers. Specific resorbable material(s) for a particular application may be selected empirically by those skilled in the art.

Preferably, the resorbable material is a carbohydrate substance. Illustrative examples of particularly preferred resorbable materials are sold under the tradenames Vicryl™ (Poly(Lactide-Co-Glycoside), a glycolic acid/lactic acid copolymer) and Dexon™. (glycolic acid polymer).

The backing material may be in the form of a solid sheet or composed of individual strands or fibers formed into a cloth or felt-like material, woven, knitted, extruded, spun, electrospun, combed, compressed or felted. Suitable pore sizes of the resulting material may be determined empirically by one of ordinary skill in the art and may range in diameter from 2000 microns to less than one nanometer. More preferably, they may range from 1000 to one microns, and more preferable from 200 to 700 microns, and most preferably from 230 to 635 microns.

In certain embodiments of the present invention the backing material may be within the mass of the haemostatic mixture. Preferably the backing material is located substantially on the side opposite the tissue-contacting face. In another preferred embodiment the backing material is located substantially within the center of the haemostatic mass.

The proteinacious components of the inventive compositions may originate in any animal species, and may be natural, modified, derivatized, recombinant, or transgenic in nature. Preferably the species of origin of naturally-derived materials is human. If the material is recombinant or transgenic in nature, preferably the species of origin of the primary amino acid sequence is human. Additional preferred species include bovine and porcine.

The fibrinogen employed in the inventive haemostatic compositions is preferably human, but any suitable preparation may be utilized. Such suitable preparations may include derivatives and metabolites, such as Fibrin I. A specific fibrinogen or fibrinogen containing composition for a particular application may be selected empirically by one of ordinary skill in the art. The fibrinogen may also contain Factor XIII at a level sufficient to produce adequate cross linking of the fibrin strands to each other, and to the tissue to which the composition is applied.

The thrombin employed in the inventive haemostatic compositions is preferably human, but any suitable preparation may be utilized. A specific thrombin or thrombin containing composition for a particular application may be selected empirically by one of ordinary skill in the art. Additionally, in each of the embodiments of the present invention, thrombin may be replaced by any of the thrombin-equivalents known by those skilled in the art to be activators of fibrinogen conversion to fibrin. Illustrative examples of such agents are snake venoms, such as batroxiben. A specific activator of fibrin formation for a particular application may be selected empirically by one skilled in the art.

In each of the embodiments of the present invention, one or more of the protein components of the composition or mixture can be coated with a slowly dissolving coat of an acceptable inactive excipient. By tailoring the composition and the thickness of the coating, the duration of partitioning of the coated component can be adjusted to coincide with the manufacturing process such that there is insufficient fibrin formation to significantly reduce the haemostatic effectiveness of the composition upon re-hydration.

Each of the inventive haemostatic bandages may also further comprise a backing material on the side of the bandage opposite the wound-facing side. The backing material may be affixed with a physiologically-acceptable adhesive or may be self-adhering (e.g by having a sufficient surface static charge). The backing material may be a resorbable material or a non-resorbable material, such as a silicone patch or plastic. Preferably, the backing material is a resorbable material.

Additionally, in each of the embodiments of the present invention, in addition to the active components of the mixture, one or more inactive carrier or filler materials may also be incorporated into the formulation. Preferred examples include albumin, Immunoglobulin, sucrose, mannitol, xylose, xylol, Chitosan and its derivatives, collagen and its derivatives, polysorbate, alginates and Fibronectin.

Additionally, in each of the embodiments of the present invention, in addition to the active components of the mixture, one or more binding materials may also be incorporated into the formulation. Preferred examples include albumin, sucrose, Chitosan and its derivatives, collagen and its derivatives, polysorbate and Fibronectin.

Additionally, in each of the embodiments of the present invention, in addition to the active components of the mixture, the composition may also optionally further comprise a release agent which may optimally be applied to the mold prior to filling with the proteinacious materials. A preferred release agent is sucrose. Others include, but are not limited to; chitosan and its derivatives, dextrose, silicone-containing compounds, detergents and oils.

Additionally, in each of the embodiments of the present invention, in addition to the active components of the mixture, one or more solubilizing materials may also be incorporated into the formulation. Preferred examples include albumin, sucrose, Chitosan and its derivatives, detergents, tensides, PEG, PPG and polysorbate.

For all of the components of the inventive embodiments, suitable materials may be obtained from various sources, and purified by any of the purification methods known to those skilled in the art. An important component of such methods include techniques that avoid, reduce or inactivate pathogens within these materials, including bacteria, molds, spores, viruses and prions.

Alternatively, a physiologically-acceptable adhesive may applied to the resorbable material and/or the backing material (when present) and the fibrinogen layer(s) and/or the thrombin layer(s) subsequently affixed thereto.

In one embodiment of the inventive bandage, the physiologically-acceptable adhesive has a shear strength and/or structure such that the resorbable material and/or backing material can be separated from the fibrinogen layer and/or the thrombin layer after application of the bandage to wounded tissue. In another embodiment, the physiologically-acceptable adhesive has a shear strength such that the resorbable material and/or backing material cannot be separated from the fibrinogen layer and/or the thrombin layer after application of the bandage to wounded tissue.

Suitable fibrinogen and thrombin may be obtained from human or mammalian plasma by any of the purification methods known and available to those skilled in the art; from supernatants or pastes of recombinant tissue culture, viruses, yeast, bacteria, or the like that contain a gene that expresses a human or mammalian plasma protein which has been introduced according to standard recombinant DNA techniques; or from the fluids (e.g, blood, milk, lymph, urine or the like) of transgenic animals that contain a gene that expresses human fibrinogen and/or human thrombin which has been introduced according to standard transgenic techniques.

As a general proposition, the purity of the fibrinogen and/or the thrombin for use in the inventive haemostatic dressing will preferably be an appropriate purity known to one of ordinary skill in the relevant art to lead to the optimal efficacy and stability of the protein. Preferably, the fibrinogen and/or the thrombin has been subjected to multiple chromatographic purification steps, such as affinity chromatography and preferably immunoaffinity chromatography, to remove substances which cause fragmentation, activation and/or degradation of the fibrinogen and/or the thrombin during manufacture, storage and/or use. Illustrative examples of such substances that are preferably removed by purification include protein contaminants, such as inter-alpha trypsin inhibitor and pre-alpha trypsin inhibitor; non-protein contaminants, such as lipids; and mixtures of protein and non-protein contaminants, such as lipoproteins.

The concentration of the fibrinogen and/or the thrombin employed in the inventive haemostatic composition or dressing is also preferably selected to optimize both the efficacy and stability thereof, as may be determined empirically by one skilled in the relevant art. During use of an inventive haemostatic bandage, the fibrinogen and the thrombin are preferably activated at the time the bandage is applied to the wounded tissue by the endogenous fluids of the patient escaping from the hemorrhaging wound. Alternatively, in situations where fluid loss from the wounded tissue is insufficient to provide adequate hydration of the protein layers, the fibrinogen and or the thrombin may be activated by a suitable, physiologically-acceptable liquid, optionally containing any necessary co-factors and/or enzymes, prior to or during application of the haemostatic bandage to the wounded tissue.

In addition, one or more supplements may also be contained in the inventive haemostatic composition, such as growth factors, drugs, polyclonal and monoclonal antibodies and other compounds. Illustrative examples of such supplements include, but are not limited to: antibiotics, such as tetracycline and ciprofloxacin, amoxicillin, and metronidazole; anticoagulants, such as activated protein C, heparin, prostacyclin (PGI.sub.2), prostaglandins, leukotrienes, antithrombin III, ADPase, and plasminogen activator; steroids, such as dexamethasone, inhibitors of prostacyclin, prostaglandins, leukotrienes and/or kinins to inhibit inflammation; cardiovascular drugs, such as calcium channel blockers, vasodilators and vasoconstrictors; chemoattractants; local anesthetics such as bupivacaine; and antiproliferative/antitumor drugs such as 5-fluorouracil (5 FU), taxol and/or taxotere; antivirals, such as gangcyclovir, zidovudine, amantidine, vidarabine, ribaravin, trifluridine, acyclovir, dideoxyuridine and antibodies to viral components or gene products; cytokines, such as .alpha.- or .beta. - or .gamma.-Interferon, .alpha.- or .beta.-tumor necrosis factor, and interleukins; colony stimulating factors; erythropoietin; antifungals, such as diflucan, ketaconizole and nystatin; antiparasitic gents, such as pentamidine; anti-inflammatory agents, such as .alpha.-1-antitrypsin and .alpha.-1-antichymotrypsin; anesthetics, such as bupivacaine; analgesics; antiseptics; and hormones. Other illustrative supplements include, but are not limited to: vitamins and other nutritional supplements; glycoproteins; fibronectin; peptides and proteins; carbohydrates (both simple and/or complex); proteoglycans; antiangiogenins; antigens; lipids or liposomes; oligonucleotides (sense and/or antisense DNA and/or RNA); and gene therapy reagents.

The following examples are illustrative only and are not intended to limit the scope of the invention as defined by the appended claims. It will be apparent to those skilled in the art that various modifications and variations can be made in the methods of the present invention without departing from the spirit and scope of the invention. Thus, it is intended that the present invention cover the modifications and variations of this invention provided they come within the scope of the appended claims and their equivalents.

### V. Examples

Methods of Rapid Freezing of a Fibrinogen and Thrombin Mixture to Minimize Fibrin Formation.

Rapid freezing of a fibrinogen/thrombin mixture can halt the chemical processes producing the formation of fibrin. The development of a rapid freezing system of a fibrinogen/thrombin mixture involves the following steps:

1. Determine the upper allowable limit of fibrin formation within the product. Gamma-gamma dimer formation, A α to A conversion, and or B β to B conversion (measures of fibrin formation) can be observed in the manufacture of the dressing. An upper limit for fibrin formation beyond which dressing performance deteriorates is established to set specifications for the mixing of fibrinogen and thrombin.

2. Develop a dispensing system for the fibrinogen and thrombin that is sufficiently rapid to limit fibrin formation to the level established in step 1 above.

3. Develop a rapidly freezing method which limit fibrin formation to a level within the fibrin specification.

The fibrinogen/thrombin mixture can also be subjected to freeze drying. This allows for room temperature storage of the product. The product can then be activated by the end user with aqueous solutions.

This process can be scaled for various size and shape molds. In this manner, this manufacturing method can be used for different products and applications.

This process can also be used as part of a high throughput system, which will reduce manufacturing costs.

Developing a Fibrin Formation Specification

A specification establishing the upper permissible limit of gamma-gamma dimer formation specification allows for rapid screening of new manufacturing procedures. The upper limit of dimer formation for the fibrin specification is set by manufacturing a fibrin sealant bandage similar to the ones known in the art, but titrating varying amounts of thrombin into the fibrinogen layers of the bandages during the manufacturing process. Overall thrombin concentration in the bandage is kept constant by decreasing t concentration of thrombin in its layer proportionally. Gamma-gamma dimer formation is determined and those particular bandages that pass QA/QC testing are then used to establish the maximum acceptable gamma-gamma dimer levels.

Alternatively, the dressings produced by the new production processes are tested to determine those that achieve suitable performance. Once these new dressings have been identified, the conditions (geometry, time, efficiency of mixing etc) of fibrinogen and thrombin mixing and freezing (temperature, mold orientation, number of cooling faces, mold material, coolant etc) that had been used are then altered to produce various levels of gamma-gamma dimer, and these levels compared with their performance in in vitro and ex vivo assays to determine an acceptable upper limit of gamma-gamma dimer, and hence fibrin, formation in the dressing.

A dispensing system for fibrinogen and thrombin.

Suitable controlled mixing/dispensing equipment is commercially available from various vendors. These systems allow for fine reproducible control of amounts of materials and speed of dispensing. Test dressings are manufactured at various pressures, orifice geometries and numbers, and flow rates to determine the optimum processes for filling the molds. Mixing of the fibrinogen and thrombin occurs as the mold is filled. Pre-chilling of the protein solutions and the rapid freezing of the materials, which will occur as or immediately after the molds are filled, limits the interaction of the fibrinogen and thrombin prior to lyophilization. While this pre-mixing may be incomplete, it should be noted that in previous fibrin sealant based haemostatic bandages, performance was not dependent upon the uniformity of fibrinogen and thrombin pre-mixing. Both a layered bandage (with minimal pre-mixing) and a powder bandage (complete premixing) produced fully functional bandages as determined in the ballistic large animal model7 and a swine aortotomy mode18.

Decreasing Fibrin Formation in the Manufacturing Process by Changing Formulations The formation of fibrin during dispensing may also be reduced by lowering the thrombin concentration in the formulation. Very high levels of thrombin were used in the previously described layered bandage because the formation of fibrin may slow the diffusion of thrombin through its matrix. This was further reinforced when interrupted thrombin layers were also examined at the ARC (WO 2004 1024195). High concentrations of thrombin were also used to insure thrombin diffusion by mass action. Diffusion issues are eliminated when the thrombin is mixed directly with the fibrinogen in the monolithic dressing. Therefore, there is no need to incorporate high concentrations of thrombin to drive diffusion. The effect is to maintain haemostatic efficacy while decreasing fibrin formation during manufacture. Thrombin concentrations are used that are low enough to keep post-manufacture fibrin levels within the fibrin specifications identified in the experiment described above.

Gradient Manufacturing Studies

A relatively high total level of fibrin formation during manufacture may be permissible as long as the concentration of fibrin is low on the surface of the dressing that faces the wound. A fibrinogen/thrombin gradient manufacturing process is employed to produce this structure. The fibrinogen and thrombin gradient is created as the mold is filled by adjusting the flow rates in such a manner that higher thrombin ratios occur on the non wound-facing side of the dressing.

By this means, various gradients can be constructed, including those with fibrinogen alone on the outer faces and fibrinogen/thrombin mixtures within, thrombin gradients that produce decreasing levels of thrombin as the wound-contacting face is approached, and those that have the opposite orientation.

A dressing is also prepared with the thrombin contained within the center of the mass of dressing material. By first dispensing fibrinogen into a horizontal mold, followed by thrombin, and finishing with another bolus of fibrinogen, a monolithic structure with the thrombin largely deposited in the center of the fibrin sealant mass is constructed. Unlike previous layered structures, there are no layers to delaminate as the material has mixed while in the liquid state prior to freezing.

Mold Orientation: Vertical Filling and Injection Molding

The dressing molds may be mounted either horizontally or vertically. The vertical orientation has the advantage of a gravity-based filling process, and two-sided cooling which reduces the amount of fibrin formation prior to freezing. There are analogous filling and freezing processes used in the food industry (ice cream bars), and thus industrial application of this process is relatively conventional.

Horizontally-oriented molds have several advantages. First, they have been used before for dressing manufacture; secondly, they can be used to manufacture the gradient-style dressing described herein, and finally, the filling and freezing of these molds will utilize technology derived from the injection-molding industry, which is designed for high throughput processing.

Development of a Rapid Freezing Method to Stop the Formation of Fibrin

Chilling rate of ⅛ inch thick protein solutions have been observed at approximately −10° C. per second when placed in contact with a steel block maintained at −60° C. The fibrinogen and thrombin are pre-cooled to −4° C. in the dispensing system and thus require less than 3 seconds to freeze. If quicker freezing times are desired, liquid nitrogen chilled blocks (−196° C.) are used. Other methods to decrease freezing time are to mount the molds vertically, between two chilled metal blocks. The vertical mounting doubles the surface area contact. A vertically mounted system has other benefits too. In the vertical system, the fibrinogen and thrombin are dispensed into the mold at minimal flow so the heat transfer rate of the system is not over-taxed. The bottom portion of the mold is frozen before the mold is completely filled.

For the vertical system, vertical slots of cGMP steel are cooled by circulating liquid nitrogen or other suitable coolant. Each square foot can accommodate eight vertical slots and therefore a 2′×6′ freezing unit is capable of freezing 96 dressings. This size of the freezing unit is selected to fit into a 3′×8′ aseptic cGMP laminar flow hood thus reducing the possibility of costly lot rejections due to product contamination.

Inhibiting Fibrin Formation During the Manufacturing Process

In another embodiment of this invention, the fibrinogen and thrombin are mixed together in a manner in which the thrombin is inhibited from reacting with the fibrinogen. This is accomplished by using a thrombin inhibitor that loses its activity when the product is used, such as the following method. Thrombin may be temporarily inhibited by manufacturing a spray dried thrombin particle coated with sucrose. The thrombin particle can be suspended in ethanol, and then the suspension is dispensed in the mold with the fibrinogen. The amount of sucrose coating can be adjusted (determined experimentally) so that it dissolves slowly enough to prevent excess fibrin formation in the brief, low temperature manufacturing of the product, but allows the sucrose/thrombin particles to dissolve within seconds when hydrated by the end user.

Making the System Size Scalable By Creating Various Size and Shape Molds.

The stations in the freezing system are designed to accommodate the largest molds (4″×4″). Smaller molds than these fit into ‘inserts’ in the 4″×4″ stations. Steel or aluminum inserts are inserted into the stations to fill the void volume and maintain a surface contact for heat transfer. The dispensing unit to be utilized is programmable and capable of dispensing any volume of material desired. Thus the system is both size and shape scalable.

Developing a High Throughput System.

The overhead dispensing unit fills 96 molds by gravity in less than 30 minutes. A horizontal dispensing (injection molding) system functions at a similar rate or greater. The slowest part of the operation is the time it takes the operator to load and unload the molds from the freezing unit. Even with this limitation, a single operator is able to produce enough dressings in one 8 hour shift to fill an industrial size freeze drier (˜1,000 4″×4″ dressings per lot). Therefore, a single small unit will suffice to manufacture ten times the annual output of the layered dressing manufacturing unit.

Inhibiting Fibrin Formation by Use of a Suitable pH

The pH of the protein solution(s) used to make the Dressing is adjusted so that it is not optimal for fibrin formation. Doing so during manufacture facilitates mixing the fibrinogen, ±factor XIII and thrombin without producing an amount of fibrin that would result in an unacceptable decreased haemostatic effectiveness. For example, thrombin has an effective pH range between 5-11, with activity significantly reduced at the extremes of this range. Thus, using a pH outside or near the extremes of its activity range greatly diminishes its activity. Products are made using a suitable buffering system, such that the pH lies outside of this range while the buffering capacity is limited, preferably significantly lower than that of blood and other bodily fluids and even intravenous resuscitation fluids, then when the Dressing is reconstituted via bodily fluids or suitable exogenous fluid(s), the buffering capacity of the reconstituting fluids will quickly readjust the products' pH to a value that permits adequate thrombin activity to yield affective product performance.

Inhibiting Fibrin Formation by Use of a Suitable Salt Concentration

The sodium concentration is adjusted so that it is not optimal for fibrin formation. In the absence of suitable sodium concentration, thrombin reverts to a form with a greatly reduced capacity for conversion of fibrinogen and Factor XIII to fibrin and Factor XIIIa respectively. When the product is used in the body, the body's own fluids, or a suitable exogenous fluid, reconstitute the bandage and convert the thrombin to a more active form that permits adequate thrombin activity to yield affective product performance.

Inhibiting Fibrin Formation by Use of a Suitable Salt Concentration and pH

As described in above, formulating the Dressing at a suitable pH or salt concentration reduces fibrin formation during manufacture, while permitting the restoration of an effective level of activity upon reconstitution with bodily fluids or a suitable exogenous fluid. A combination of both these strategies is even more effective. Thus when the product is used in the body, the body's own fluids, or a suitable exogenous fluid, will reconstitute the bandage and convert the thrombin to a more active form that permits adequate thrombin activity to yield affective product performance.

Inhibiting Fibrin Formation by an Absence of Ca+2 and/or Mg+2

The presence of Calcium and/or Magnesium ions are required for the activation of prothrombin to thrombin and for the optimal activity of Factor XIIIa. Both these ions are commonly found in most bodily fluids, including blood. Therefore the manufacture of the Dressing using solutions devoid of or with greatly reduced levels of one or both of these ions permits the mixing of the reagents, without excessive fibrin formation. When the product is used in the body, the body's own fluids, or a suitable exogenous fluid, will reconstitute the bandage with a fluid or fluids containing adequate levels of one or both of these ions to convert the thrombin and/or Factor XIII to a more active form that permits adequate activity to yield effective product performance.

This invention describes a method to mix aqueous fibrinogen, ±factor XIII and thrombin together to form a single mass, under conditions that limit the formation of fibrin. The method employs rapid freezing of the components after mixing to limit fibrin formation. The fibrinogen/Factor XIII and thrombin are kept in separate dispensing units as long as possible. The components are mixed at the nozzles of the dispensing units and are dispensed at a slow enough rate into a casting mold at maintained at freezing temperatures in such a manner that the components freeze on contact with the casting mold.

Since thrombin catalyzes the formation of fibrin, it is necessary to adjust the thrombin concentration in the mixture. The thrombin concentration can always be adjusted low enough to limit fibrin formation, but at very low concentrations there may not be enough thrombin to catalyze fibrin formation by the user of the product. Therefore, the thrombin concentration has to be determined experimentally to establish a concentration that limits fibrin formation during the manufacturing process and yet is sufficiently high enough to be useful to the user of the product.

Inhibiting Fibrin Formation by Use of a Suitable pH with a Volatile Buffer

The pH of the protein solution(s) used to make the Dressing is adjusted with volatile buffer salts so that it is not optimal for fibrin formation during preparation of the mixture, but becomes optimal pH for fibrin formation after lyophilization, during which process the volatile buffer(s) is removed by evaporation. Examples of volatile buffers are ammonium acetate used between pH 4 and pH 7 (Doolittle, Biochem J 94: 742, 1965) and ammonium bicarbonate at pH 8-9. (http://www.molecularinfor.com/MTM/G/G3/G3-1/G3-1-7.html).

Inhibiting Fibrin Formation by Use of Chaotropic Compounds

The protein solution(s) used to make the Dressing includes chaotropic salts that diminish fibrin formation during preparation of the mixture. It is understood that such chaotropic compounds are to be used in conjunction with other conditions (pH, temperature, other formulation components, protein concentration) to achieve an optimal condition.

1. A solid dressing for treating wounded tissue in a mammal, said solid dressing comprising at least one haemostatic layer having a wound facing surface and an opposite surface, and consisting essentially of fibrinogen and a solvent consisting of water and a fibrinogen activator, wherein said haemostatic layer is substantially homogenous, and wherein said fibrinogen is present in an amount about 13.0 mg/cm² of the wound facing surface of said dressing, and wherein the moisture content of said solid dressing is from 6% to 44%.

2. The solid dressing of claim 1, further comprising at least one support layer.

3. The solid dressing of claim 2, wherein said support layer comprises a backing material.

4. The solid dressing of claim 1, wherein said haemostatic layer also contains a fibrin cross-linker and/or a source of calcium ions.

5. The solid dressing of claim 1, wherein said haemostatic layer also contains one or more of the following: at least one filler, at least one solubilizing agent, at least one foaming agent and at least one release agent.

6. The solid dressing of claim 1, wherein said haemostatic layer is cast as a single piece.

7. The solid dressing of claim 1, wherein said haemostatic layer is composed of a plurality of particles, each of said particles consisting essentially of fibrinogen and thrombin.

8. The solid dressing of claim 7, wherein said haemostatic layer further contains at least one binding agent in an amount effective to improve the adherence of said particles to one another.

9. The solid dressing of claim 1, wherein said haemostatic layer is a monolith.

10. The solid dressing of claim 1, wherein said haemostatic layer has been lyophilized.

11. The solid dressing of claim 1, wherein said haemostatic layer is substantially free of fibrin.

12. A solid dressing for treating wounded tissue in a mammal comprising at least one haemostatic layer consisting essentially of thrombin and a fibrinogen component, wherein said thrombin is present in an amount between about 0.250 Units/mg of fibrinogen component and 0.062 Units/mg of fibrinogen component, wherein said haemostatic layer is composed of a plurality of particles, each of said particles consisting essentially of fibrinogen and thrombin, and wherein said haemostatic layer is substantially homogenous and frozen.

13. The solid dressing of claim 12, wherein said support layer comprises a backing material.

14. The solid dressing of claim 13, further comprising at least a physiologically acceptable adhesive between said haemostatic layer and said backing layer.

15. The solid dressing of claim 12, wherein said haemostatic layer also contains at least one therapeutic supplement selected from the group consisting of antibiotics, anticoagulants, steroids, cardiovascular drugs, growth factors, antibodies (poly and mono), chemoattractants, anesthetics, antiproliferatives/antitumor agents, antivirals, cytokines, colony stimulating factors, antifungals, antiparasitics, anti-inflammatories, antiseptics, hormones, vitamins, glycoproteins, fibronectin, peptides, proteins, carbohydrates, proteoglycans, antiangiogenins, antigens, nucleotides, lipids, liposomes, fibrinolysis inhibitors and gene therapy reagents.

16. The solid dressing of claim 12, wherein said mammalian fibrinogen is present in an amount between 1.5 mg/cm² of the wound-facing surface of said dressing and 13.0 mg/cm² of the wound-facing surface of said dressing.

17. A haemostatic composition comprising a frozen mixture of fibrinogen and thrombin, with or without Factor XIII, which contains insufficient fibrin to prohibit its effective use as a haemostatic agent, and which further retains the ability to convert sufficient fibrinogen to fibrin upon thawing to provide effective hemostasis.

18. The composition of claim 17, wherein the composition also contains one or more of the following: at least one foaming agents, at least one filler material, at least one binding material, at least one solubilizing agents, and at least one release agents.

19. The composition of claim 17, wherein any of the proteinaceous components may originate in an animal species such as human, porcine, bovine, equine, caprine and piscine.

20. The composition of claim 17, wherein the composition further comprises one or more drugs or biologicals of therapeutic use.
